(19) United States
(12) Patent Application Publication
(10) Pub. No.: US 2024/0341839 A1
(43) Pub. Date: Oct. 17, 2024

(54) THERMAL MODULATION AND DETECTION OF PERIVASCULAR TISSUES

(71) Applicant: Medtronic Ireland Manufacturing Unlimited Company, Dublin 2 (IE)

(72) Inventors: Douglas A. Hettrick, Andover, MN (US); Paul J. Coates, Corte Madera, CA (US)

(21) Appl. No.: 18/294,711

(22) PCT Filed: Aug. 12, 2022
(86) PCT No.: PCT/EP2022/072710

§ 371 Date: Feb. 2, 2024

Related U.S. Application Data
(60) Provisional application No. 63/232,434, filed on Aug. 12, 2021.

(51) Int. Cl. A61B 18/14; A61B 18/00

(52) U.S. Cl. CPC A61B 18/1492; A61B 2018/00345; A61B 2018/00791; A61B 2018/00875

(57) ABSTRACT

An intravascular medical device includes a support structure, a plurality of focal energy sources, and a plurality of temperature sensors. The support structure defines a longitudinal axis and is configured to ‘be positioned within a vessel of a patient. The plurality of focal energy sources is arranged around a perimeter of the support structure. Each of the plurality of focal energy sources is configured to deliver energy to one or more perivascular tissues near the vessel to heat the one or more perivascular tissues. The plurality of temperature sensors is arranged around the perimeter of the support structure. Each of the plurality of temperature sensors is configured to measure a temperature at or near a wall of the vessel.

### TECHNICAL FIELD

The present technology is related to mapping of perivascular tissues. In particular, various examples of the present technology are related to devices and systems for thermal modulation and detection of perivascular tissues.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function can be considerably stimulated in disease states characterized by heightened sympathetic tone. For example, reduced renal blood flow and glomerular filtration rate as a result of renal sympathetic efferent stimulation may be a cornerstone of the loss of renal function in cardio-renal syndrome, (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, can have significant limitations including limited efficacy, compliance issues, side effects, and others.

### SUMMARY

The present technology is directed to devices, systems, and methods for thermal modulation and detection of perivascular tissues.

In some examples, the disclosure describes an intravascular medical device that includes a support structure, a plurality of focal energy sources arranged around a perimeter of the support structure, and a plurality of temperature sensors arranged around the perimeter of the support structure. The support structure defines a longitudinal axis and is configured to be positioned within a vessel of a patient. Each of the plurality of focal energy sources is configured to deliver energy to one or more perivascular tissues near the vessel to heat the one or more perivascular tissues. Each of the plurality of temperature sensors is configured to measure a temperature at or near a wall of the vessel.

In some examples, the disclosure describes an intravascular medical device system includes a first intravascular medical device and a second intravascular medical device. The first intravascular medical device includes a first support structure and an energy source coupled to the first support structure. The first support structure defines a longitudinal axis and is configured to be positioned within a first vessel of a patient. The energy source is configured to deliver energy to one or more perivascular tissues near the first vessel to heat the one or more perivascular tissues. The second intravascular medical device includes a second support structure and a plurality of temperature sensors arranged around the perimeter of the second support structure. The second support structure defines a longitudinal axis and is configured to be positioned within the same first vessel or a different second vessel of a patient. Each of the plurality of temperature sensors is configured to measure a temperature at or near a wall of the second vessel.

In some examples, the disclosure describes a tissue mapping system that includes an intravascular medical device and a tissue mapping device. The intravascular medical device includes a support structure and a plurality of temperature sensors arranged around a perimeter of the support structure. The support structure defines a longitudinal axis and configured to be positioned within a vessel of a patient. Each of the plurality of temperature sensors corresponds to a particular axial and circumferential position on the support structure, and each of the plurality of temperature sensors is configured to measure a temperature at or near the wall of the vessel. The tissue mapping device is configured to receive a temperature signal that includes a temperature measurement from each of the plurality of temperature sensors. Each temperature measurement represents the temperature of the wall of the vessel at the respective axial and circumferential position on the support structure.

In some examples, the disclosure describes a method that includes modulating a temperature of one or more perivascular tissues near a vessel of a patient and detecting, using an intravascular medical device positioned in the vessel, a spatial or temporal distribution of temperatures at or near a wall of the vessel. The intravascular medical device includes a support structure defining a longitudinal axis and a plurality of temperature sensors arranged around a perimeter of the support structure.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout and wherein:

FIG. 1A is a partially schematic illustration of a thermal modulation and detection system configured in accordance with some examples of the present disclosure.

FIG. 1B is a schematic and conceptual illustration of an example programmer coupled to an imaging system and an example therapy delivery device in accordance with some examples of the present disclosure.

FIG. 2A is a perspective view conceptual illustration of a distal portion of an example intravascular medical device shown in FIG. 1A, with a vessel shown in axial cross-section.

FIG. 2B is a side view conceptual illustration of a distal portion of an example intravascular medical device shown in FIG. 1A.

FIG. 3A is an expanded side view conceptual illustration of a distal portion of an example intravascular medical device in accordance with some examples of the present disclosure.

FIG. 3B is an expanded side view conceptual illustration of a distal portion of an example intravascular medical device in accordance with some examples of the present disclosure.

FIG. 3C is an expanded side view conceptual illustration of a distal portion of an example intravascular medical device in accordance with some examples of the present disclosure.

FIG. 3D is an expanded side view conceptual illustration of a distal portion of an example intravascular medical device assembly in accordance with some examples of the present disclosure.

FIG. 4A is a cross-sectional illustration of perivascular tissue near a renal artery.

FIG. 4B is a cross-sectional illustration of the perivascular tissue of FIG. 3A that includes a thermal field generated from an intravascular medical device in accordance with some examples of the present disclosure.

FIG. 5A-5D are illustrations of thermal fields generated by an intravascular medical device from various electrodes positioned along a vessel at various circumferential locations in accordance with some examples of the present disclosure.

FIG. 6A is a cross-sectional illustration of perivascular tissue near a renal artery.

FIG. 6B-6C are illustrations of thermal fields generated by an intravascular medical device from various electrodes positioned along a vessel at various axial locations in accordance with some examples of the present disclosure.

FIG. 7A is a flowchart of an example method for generating and detecting a thermal field in perivascular tissue in accordance with some examples of the present disclosure.

FIG. 7B is a flowchart of an example method for controlling an ablation catheter in accordance with some examples of the present disclosure.

FIG. 7C is a flowchart of an example method for controlling an ablation catheter in accordance with some examples of the present disclosure.

FIG. 8A-8E are illustrations of an energy field and corresponding graph delivered to different tissues at different distances in accordance with some examples of the present disclosure.

### DETAILED DESCRIPTION

The present technology is directed to devices, systems, and methods for thermal modulation and detection of perivascular tissues, such as renal perivascular tissues.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

Renal neuromodulation, such as renal denervation, may be used to modulate activity of one or more renal nerves and affect activity of the sympathetic nervous system (SNS). In renal neuromodulation, therapeutic elements may be introduced near renal nerves located between an aorta and a kidney of a patient. Renal neuromodulation may be accomplished using one or more of a variety of treatment modalities, including electrical stimulation, radio frequency (RF) energy, microwave energy, ultrasound energy, a chemical agent, or the like. For example, an RF ablation system may include an RF generator configured to generate RF energy and deliver RF energy to perivascular tissues via one or more electrodes carried by a catheter and positioned within a lumen of a body of a patient. The RF energy may heat tissue to which the RF energy is directed (which tissue includes one or more renal nerves) and modulate the activity of the one or more renal nerves. In many patients, renal nerves generally follow the renal artery and branch vessels from near the aorta to a kidney, and may be present in perivascular tissues surrounding the renal artery and/or branch vessels. Because renal nerves may be around the renal artery and/or branch vessels and may include multiple nerves and/or nerve branches, RF energy may be delivered around the renal artery and/or branch vessels to affect as many renal nerves as possible. However, arrangement of perivascular tissues may vary between patients, such that RF energy applied at a particular location in the renal artery may produce different lesions and affect perivascular tissues in different ways from one patient to the next.

In accordance with the current disclosure, an intravascular medical device (e.g., an RF ablation catheter) may be configured to detect spatial and/or temporal variations in temperature of perivascular tissues in a patient to more efficiently ablate particular tissues. The intravascular medical device includes a support structure and temperature sensors arranged around the perimeter of the support structure. Each of the temperature sensors is configured to measure a temperature at or near the wall of a vessel in which the intravascular medical device is positioned. In some examples, an energy source, such as RF electrodes arranged around the perimeter of the support structure, delivers energy to or removes energy from the perivascular tissues near the vessel to modulate the temperature of the perivascular tissues. For example, the energy source may apply RF or ultrasound energy to the perivascular tissues from within the vessel or apply RF energy to the perivascular tissues from another adjacent vessel, any of which may modulate a temperature of perivascular tissues. In other examples, a temperature modulation device other than an energy source, such as a flow restricting device, may control an amount of heat delivered to or removed from the perivascular tissues, such as by temporarily restricting the flow of fluid through convective tissues, such as vessels, of the perivascular tissues.

Perivascular tissue is heterogenous, and includes different combinations of tissues at different locations within a volume near a renal artery. As such, particular perivascular tissues may heat or cool differently based on various properties of the tissue, various locations of the tissue, and various relationships of the tissue to the energy source, such as a heat capacity of the tissue, a flow capacity of the tissue, or a distance of the tissue from the energy source. The temperatures measured by the temperature sensors at or near the wall of the vessel may reflect the spatial and/or temporal variation in temperature of the surrounding perivascular tissues and provide an indication, alone or in combination with impedance information from the perivascular tissues, as to a relative location of particular perivascular tissues, (e.g., renal nerves) or of perivascular tissues conducive to receiving an RF field (e.g., non-convective tissues that draw less heat away). By determining a relative location of perivascular tissues in this manner, energy may be delivered to perivascular tissues corresponding to particular circumferential or axial locations of the vessel that may be more likely to include target tissues (e.g., renal nerves) or may produce a greater temperature increase for an amount of energy applied, which may reduce a likelihood of renal nerves being left untreated, reduce an amount energy applied to the perivascular tissues, and/or improve a likelihood of success of the denervation therapy.

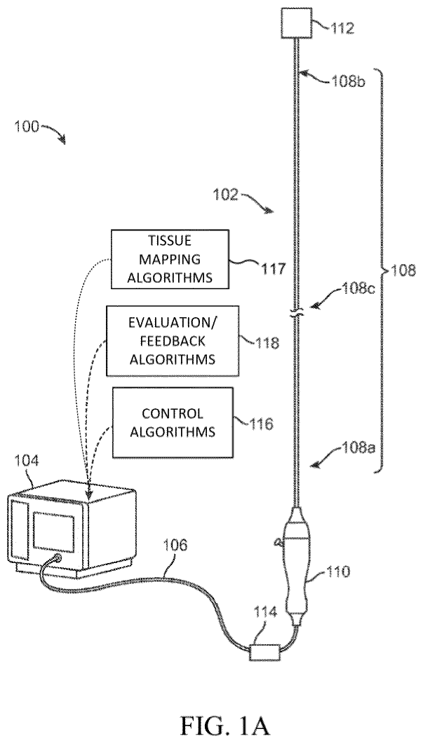

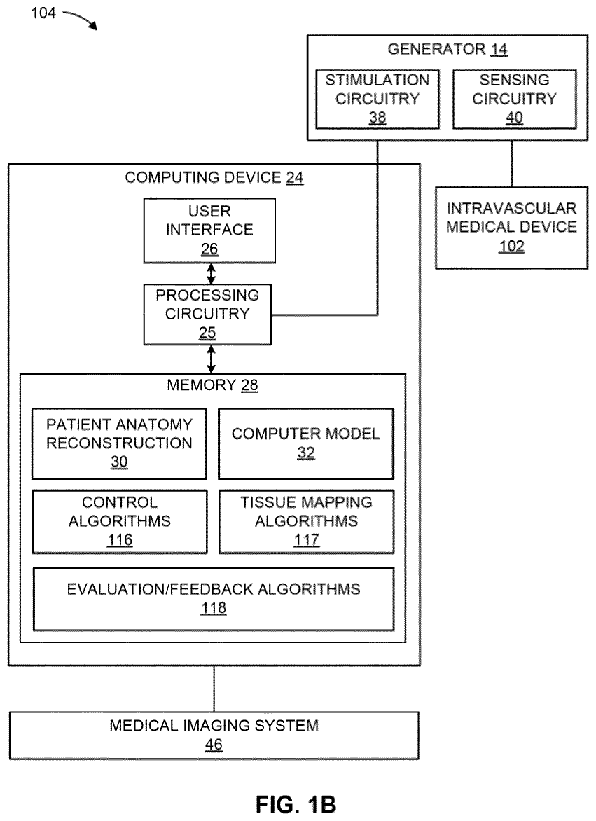

FIG. 1A is a partially schematic perspective view illustrating a thermal modulation and detection system 100 configured in accordance with some examples of the present disclosure. System 100 may include an intravascular medical device 102, a control system 104, and a cable 106 extending between intravascular medical device 102 and control system 104. Intravascular medical device 102 may include a shaft 108 having a proximal portion 108a, a distal portion 108b, and an optional intermediate portion 108c between proximal portion 108a and distal portion 108b. Intravascular medical device 102 may further include a handle 110 operably connected to shaft 108 via proximal portion 108a and a thermal modulation and detection element 112 (shown schematically in FIG. 1) that is part of or attached to distal portion 108b. Shaft 108 may be configured to locate thermal modulation and detection element 112 at a location within or otherwise proximate to a body lumen (e.g., a blood vessel, a duct, an airway, or another naturally occurring lumen within the human body). In some examples, shaft 108 may be configured to locate thermal modulation and detection element 112 at an intraluminal (e.g., intravascular) location. Shaft 108 and thermal modulation and detection element 112 may measure 2, 3, 4, 5, 6, or 7 French or other suitable sizes.

Intraluminal delivery of intravascular medical device 102 may include percutaneously inserting a guidewire (not shown) into a body lumen of a patient and moving shaft 108 and thermal modulation and detection element 112 along the guide wire until thermal modulation and detection element 112 reaches a suitable treatment location. Alternatively, intravascular medical device 102 may be a steerable or non-steerable device configured for use without a guide wire. Additionally, or alternatively, intravascular medical device 102 may be configured for use with a guide catheter or sheath, alone, or in addition to a guidewire.

Control system 104 is configured to control, monitor, supply, and/or otherwise support operation of intravascular medical device 102. In other examples, intravascular medical device 102 may be self-contained or otherwise configured for operation independent of control system 104. When present, control system 104 may be configured to generate a selected form and/or magnitude of energy for delivery to tissue at a treatment location via thermal modulation and detection element 112. For example, control system 104 may include an RF generator configured to generate radio frequency (RF) energy (e.g., monopolar and/or bipolar RF energy). In other examples, control system 104 may include another type of device configured to generate and deliver another suitable type of energy to thermal modulation and detection element 112 for delivery to perivascular tissue at a treatment location via one or more energy sources (not shown) of thermal modulation and detection element 112. In some examples, such energy sources may include one or more discrete, focal energy sources, such as electrodes, configured to deliver energy to the perivascular tissue at particular locations. In some examples, such energy sources may include one or more dispersed energy sources, such as a heated balloon, configured to deliver energy to the perivascular tissue in a relatively uniform manner. Along cable 106 or at another suitable location within system 100, system 100 may include a control device 114 configured to initiate, terminate, and/or adjust operation of one or more components of intravascular medical device 102 directly and/or via control system 104. In the example of FIG. 1, control system 104 is configured to receive temperature measurements, and optionally impedance measurements, from thermal modulation and detection element 112.

Thermal modulation and detection element 112 may be configured to provide or support thermal mapping or a thermal treatment (e.g., neuromodulation) at a mapping or treatment location. While described as providing thermal modulation and detection through a single thermal modulation and detection element 112, in some examples, thermal modulation and detection element 112 may be include multiple elements, such as a thermal modulation element for delivering energy to perivascular tissues and a thermal detection element for detecting temperatures caused by temperature variations in perivascular tissues.

Thermal modulation and detection element 112 may be configured to position within the vessel near the target perivascular tissues, receive energy from control system 104, and deliver the energy to the perivascular tissues. Based on thermal, electrical, and/or convective properties and relative location of the perivascular tissues, the perivascular tissues may heat to different temperatures and/or at different rates. The temperature at the wall of the vessel may reflect the temperature response in the perivascular tissues caused by the different properties and the relative location of the perivascular tissues. Thermal modulation and detection element 112 may be configured to measure the temperature at various axial and/or circumferential locations on the wall of the vessel to detect a spatial and/or temporal distribution of the temperature. Thermal modulation and detection element 112 may send thermal data representing the spatial and/or temporal distribution of temperature to control system 104, or another computing device.

FIG. 1B is a schematic and conceptual illustration of example control system 104 that includes a computing device 24 and an energy field generator 14 coupled to a medical imaging system 46 and example intravascular medical device 102. While various circuitries, algorithms, modules, and functions are described with reference to computing device 24 of FIG. 1B, in other examples, generator 14, or another medical device may include features and perform functions described with reference to computing device 24.

Computing device 24 includes processing circuitry 25, a user interface 26, and a memory 28. Memory 28 includes computer-readable instructions that, when executed by processing circuitry 25, causes computing device 24 to perform various functions. Processing circuitry 25 may include any one or more microprocessors, controllers, digital signal processors (DSPs), application specific integrated circuits (ASICs), field-programmable gate arrays (FPGAs), or equivalent discrete or integrated digital or analog logic circuitry, and the functions attributed to processing circuitry 25 herein may be embodied as software, firmware, hardware or any combination thereof.

Memory 28 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Memory 28 may store any suitable information, including patient identification information, and information for generating one or more therapy program with which generator 14 generates and delivers denervation therapy to a patient. For example, memory 28 may store one or more of patient anatomy reconstruction 30, computer model 32, control algorithms 116, tissue mapping algorithms 117, evaluation/feedback algorithms 118, and operating instructions in separate memories within memory 28 or separate areas within memory 28.

Processing circuitry 25 may be configured to develop computer model 32 based on patient anatomy reconstruction 30, tissue mapping algorithms 117, and thermal data from intravascular medical device 102. In some examples, digital reconstruction 30 includes a three-dimensional (3D) reconstruction. For example, patient anatomy reconstruction 30 may include classification data used to classify one or more perivascular tissues based on thermal data and/or thermal maps determined from thermal data.

Control algorithms 116 may define a particular program of therapy in terms of respective values for stimulation parameters, such as the one or more focal energy sources, with which the stimulus is delivered to a patient, such as electrode polarity (if applicable), duty cycle, current or voltage amplitude, and/or frequency, or appropriate non-electrical parameters in examples in which the denervation stimulus includes non-electrical stimulus. Memory 28 may also store operating instructions with which processing circuitry 25 controls the operation of computing device 24.

Generator 14 is configured to receive one or more control algorithms 116 from computing device 24, and apply one or more therapy parameter values specified by the received control algorithms 116, such as temperature or electrical parameters such as amplitude, duty cycle, and frequency, to generate an energy field. For example, generator 14 may control stimulation circuitry 38 to generate a stimulation signal according to a particular control algorithm, and deliver the stimulation signal via intravascular medical device 102. Stimulation circuitry 38 may be communicatively coupled to the one or more conductors of intravascular medical device 102 using any suitable technique. For example, generator 14 may include switching circuitry configured to switch the stimulation generated by stimulation circuitry 38 across different energy sources, such as electrodes or other focal energy sources, or generator 14 may include multiple energy sources to drive more than one electrode at one time. In some examples, generator 14 (or computing device 24 or other device) may include sensing circuitry 40 coupled to intravascular medical device 102 and configured to receive measurements, feedback, or signals, such as temperature signals from temperature sensors or impedance signals from electrodes.

A user, such as a clinician, may interact with processing circuitry 25 through user interface 26. User interface 26 may include a display, such as a liquid crystal display (LCD), light-emitting diode (LED) display, or other screen, to present information related to stimulation therapy, and buttons or a pad to provide input to computing device 24. In examples in which user interface 26 requires a 3D environment, the user interface may support 3D environments such as a holographic display, a stereoscopic display, an autostereoscopic display, a head-mounted 3D display, or any other display that is capable of presenting a 3D image to the user. Buttons of user interface 26 may include an on/off switch, plus and minus buttons to zoom in or out or navigate through options, a select button to pick or store an input, and pointing device, e.g. a mouse, trackball, or stylus. Other input devices may be a wheel to scroll through options or a touch pad to move a pointing device on the display. In some examples, the display may be a touch screen that enables the user to select options directly from the display screen.

In some examples, computing device 24 may include a telemetry module that may support wired or wireless communication between computing device 24 and generator 14 or another computing device under the control of processing circuitry 25. A clinician or another user may interact with computing device 24 to generate and/or select control algorithms 116 for delivery via therapy delivery device 12. In some examples, computing device 24 may allow a clinician to define target volumes of influence, and generate appropriate therapy delivery parameter values to achieve the desired volumes of influence. Computing device 24 may be used to present anatomical regions to the clinician via user interface 26, select control algorithms 116, generate new control algorithms, and communicate the selected control algorithms 116 to the generator 14.

In some examples, computing device 24 may be communicatively coupled to medical imaging system 46, or may otherwise receive one or more medical images of a patient from medical imaging system 46. Medical imaging system 46 may be configured to generate a medical image of a region of a patient that includes a treatment site (e.g., intended to be denervated) and, in some cases, a corresponding blood vessel. The corresponding blood vessel may be, for example, an artery or another blood vessel through which the treatment site may be accessed by intravascular medical device 102. One or more medical images generated by medical imaging system 46 may be stored by computing device 24 in memory 28, or otherwise used by processing circuitry 25, to generate patient anatomy digital reconstruction 30, such as in combination with thermal data generated by intravascular medical device 102. In some examples, medical imaging system 46 includes at least one of a fluoroscopy system, a computer aided tomography (CAT) scan system, a magnetic resonance imaging (MRI) system, a positron emission tomography (PET) scan system, an electrical impedance tomography (EIT) system, an ultrasound system, or an optical imaging system.

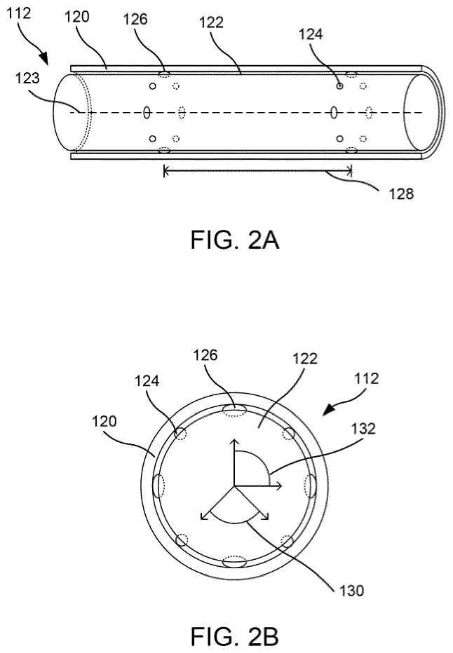

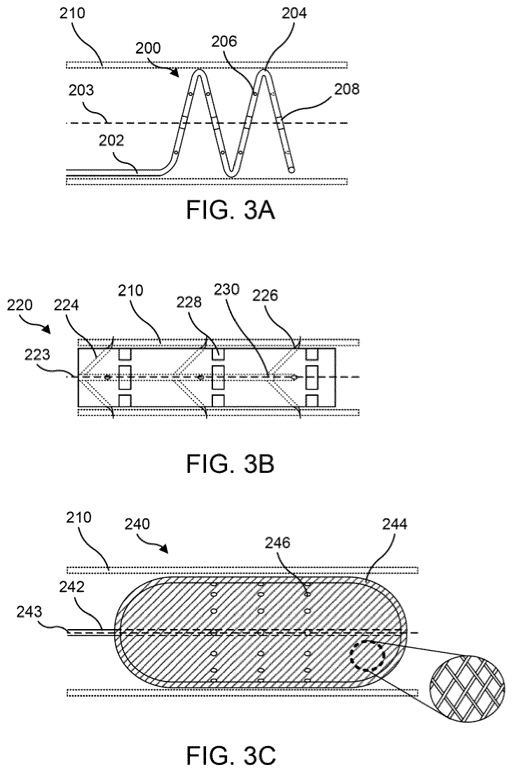

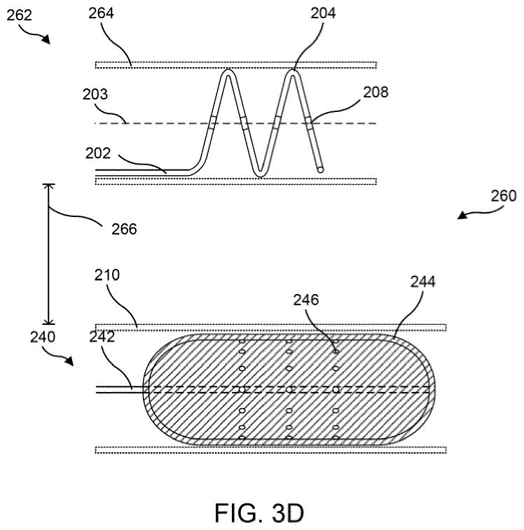

FIG. 2A is a perspective view conceptual illustration of distal portion 108b of an example intravascular medical device 102 shown in FIG. 1A, with a vessel shown in axial cross-section, while FIG. 2B is a side view conceptual illustration of distal portion 108b of an example intravascular medical device 102 shown in FIG. 1A that includes thermal modulation and detection element 112. Thermal modulation and detection element 112 includes a support structure 122 defining a longitudinal axis 123 and configured to be positioned within a vessel 120 of a patient. In some examples, support structure 122 may be a continuation of shaft 108, while in other examples, support structure 122 may be a separate structure coupled to a distal end of shaft 108. For purposes of illustration, support structure 122 is illustrated as a cylinder having a diameter only slightly smaller than a diameter of vessel 120; however, in other examples, such as shown in FIGS. 3A-3D, support structure 122 may have any of a variety of shapes and may have a diameter that is variable or expandable to the diameter of vessel 120. In some examples, support structure 122 includes an ablation catheter.

Thermal modulation and detection element 112 includes a plurality of focal energy sources 126 arranged around a perimeter and along an axis of support structure 122 at spaced-apart locations. As shown, focal energy sources 126 are arranged at different axial positions along support structure 122 according to an axial spacing and arranged at different circumferential position around support structure 122 according to a circumferential spacing. For example, an axial spacing 128 or circumferential spacing of adjacent focal energy sources 126 may be less than about 10 mm, while an angular spacing 132 of adjacent focal energy sources 126 around the perimeter of support structure 122 may be less than or equal to about 90 degrees. Thermal modulation and detection element 112 may include any suitable number of focal energy sources 126. The number of electrodes may be selected based on one or more of a variety of factors, including, for example, a number of channels provided by an energy field generator of control system 104 (FIG. 1), desired flexibility of distal portion 108b, desired continuity (e.g., circumferential continuity) or shape of the energy field delivered by focal energy sources 126, or the like. Focal energy sources 126 are electrically coupled to control system 104 of FIG. 1A through one or more electrical or thermal conductors (not shown). In some examples, each of focal energy sources 126 may be independently operable, such that different amounts and/or frequencies of energy may be delivered from particular focal energy sources 126 and, correspondingly, particular circumferential locations of element 112.

Each of the plurality of focal energy sources 126 is configured to deliver energy to one or more perivascular tissues of a patient to heat the perivascular tissues. Perivascular tissues may include tissues surrounding vessel 120, such as arteries, veins, lymph nodes, muscles, and other tissues. In some instances, a perivascular tissue may be any tissue that is within an effective range of an ablation catheter, such as within about two centimeters in a radial direction of a vessel in which medical device 102 is positioned. A variety of focal energy sources may be used including, but not limited to, electrodes to delivery electromagnetic energy or electric current, heating elements configured to deliver thermal energy, piezoelectric elements configured to deliver ultrasound energy, or other energy sources configured to deliver energy. In some examples, the plurality of focal energy sources 126 includes a plurality of radiofrequency electrodes.

In some examples, at least a portion of focal energy sources 126 may be configured to operate as impedance electrodes. For example, at least a portion of focal energy sources 126 may be configured to deliver a current to the wall of vessel 120 and measure an impedance from the wall of vessel 120 based on the current and voltage. The impedance at the wall of vessel 120 may represent an impedance of one or more perivascular tissues.

Thermal modulation and detection element 112 includes a plurality of temperature sensors 124 arranged around the perimeter and along the axis of support structure 122 at spaced-apart locations. The plurality of temperature sensors 124 may be present in an arrangement to provide a particular resolution. As one example, the plurality of temperatures sensors 124 may have a relatively large spacing to produce a low resolution, such as for providing local feedback as to the degree to which an electrode adjacent to a particular temperature sensor is heating an adjacent tissue. As another example, the plurality of temperature sensors 124 may have a relatively small spacing to produce a high resolution, such as for determining a relative position of particular tissues.

As shown, temperature sensors 124 are arranged at various axial positions along support structure 122 according to a distance spacing and arranged at different circumferential position around support structure 122 according to an angular spacing 130. For example, the axial spacing 128 or a circumferential spacing of adjacent temperature sensors 124 may be less than about 5 mm, such as less than 1 mm for high resolutions, while an angular spacing 130 of adjacent temperature sensors 124 around the perimeter of support structure 122 may be less than or equal to about 90 degrees, such as less than or equal to about 45 degrees for high resolution. While shown as having a same axial and angular spacing as the plurality of focal energy sources 126, the plurality of temperature sensors 124 may have any spacing. Thermal modulation and detection element 112 may include any suitable number of temperature sensors 124. The number of temperature sensors may be selected based on one or more of a variety of factors, including, for example, a number of channels received by control system 104 (FIG. 1), desired flexibility of distal portion 108b, desired continuity (e.g., circumferential continuity) or resolution of the thermal field detected by temperature sensors 124, or the like. Temperature sensors 124 are electrically coupled to control system 104 of FIG. 1A through one or more electrical conductors (not shown).

Each of the plurality of temperature sensors 124 is configured to measure a temperature at or near the wall of vessel 120. For example, each temperature sensor 124 may be positioned within about 1 millimeter of the wall of vessel 120 within vessel 120 or within about 2 millimeters of the wall of vessel 120 external to vessel 120. In some examples, each of the plurality of temperature sensors 124 is configured to contact the wall of vessel 120. For example, temperature sensors 124 may contact an inner surface of the wall of vessel 120 or extend through the wall of vessel 120. In some examples, support structure 122 may be configured to radially extend temperature sensors 124, such that each temperature sensor 124 contacts and is in thermal communication with the inner wall of vessel 120. For example, support structure 122 may be configured to radially expand, aided or unaided, from a delivery configuration to a deployed configuration to position temperature sensors 124 against the wall of vessel 120 and cause temperature sensors 124 to contact the wall of vessel 120.

In some examples, each of the plurality of temperature sensors 124 may correspond to a particular axial and circumferential position on support structure 122. Each temperature measurement by a particular temperature sensor 124 may represent the temperature of the wall of vessel 120 at the respective axial and circumferential position of the particular temperature sensor 124 on support structure 122. This respective axial and circumferential position may correspond to an axial and circumferential position within vessel 120. For example, prior to a tissue mapping or ablation procedure, an axial and circumferential position of a distal portion of intravascular medical device 102 within vessel 120 may be determined, such as through imaging, and operate as a reference point for associating an axial and circumferential position of a particular temperature sensors 124 with a particular axial and circumferential position of vessel 120. Intravascular medical device 102 may be configured to output a temperature signal that includes a temperature measurement from each of the plurality of temperature sensors 124.

Referring back to FIG. 1, control system 104 is communicatively coupled to the plurality of focal energy sources 126 and configured to control the plurality of focal energy sources 126 to deliver the energy to the perivascular tissues. Control system 104 may be configured to execute an automated control algorithms 116 and/or to receive control instructions from an operator. Control algorithms 116 may be configured to deliver energy to particular focal energy sources 126 at a particular time. For example, control algorithm 116 may deliver energy to particular focal energy sources 126 associated with particular circumferential locations within vessel 120. The plurality of focal energy sources 126 may, in combination, generate an energy field in the perivascular tissue.

Control system 104 may be configured to control an amount or type of energy delivered via the plurality of focal energy sources 126 to produce an amount of heat in the perivascular tissue. In some examples, control system 104 is configured to, in an imaging mode, control the plurality of focal energy sources 126 to heat the perivascular tissues below an ablation temperature of the perivascular tissues. Prior to performing an ablation procedure, a clinician may operate control system 104 to map the perivascular tissues in the imaging mode using temperatures below a therapeutic dose sufficient to cause ablation.

In some examples, control system 104 is configured to, in an ablation mode, control the plurality of electrodes to heat the one or more tissues above an ablation temperature of the one or more tissues. Control system 104 may be configured to provide feedback to an operator before, during, and/or after an ablation procedure via evaluation/feedback algorithms 118. During a first iteration of an ablation procedure, the clinician may operate control system 104 to ablate the perivascular tissues and, based on temperature feedback received from the plurality of temperature sensors 124, adjust position or operation of intravascular medical device 102 in one or more subsequent iterations. In some examples, control system 104 may be configured to automatically adjust operation of the plurality of focal energy sources 126. For example, control system 104 may be configured to receive a temperature signal from intravascular medical device 102 that includes a temperature measurement from each of the plurality of temperature sensors 124 and determine, based on the temperature signal, a modification to the energy delivered to at least one electrode of the plurality of focal energy sources 126.

Control system 104 may be configured to map one or more tissues via tissue mapping algorithms 117. Tissue mapping algorithms 117 may be configured to use the thermal data from intravascular medical device 102 for mapping and/or classifying perivascular tissues. Control system 104 may be configured to receive a temperature signal that includes a temperature measurement from each of the plurality of temperature sensors 124. Each temperature measurement represents the temperature of the wall of vessel 120 at the respective axial and circumferential position on support structure 122. Control system 104 may be configured to generate, based on the one or more temperature measurements, thermal data representing a spatial or temporal temperature distribution of the wall of the vessel at the respective axial and circumferential position on support structure 122. Control system 104 may further process this thermal data to determine a spatial temperature distribution (e.g., a thermal field map) of the perivascular tissues, such as by consolidating different temperature distributions generated by different energy fields produced by varying electrodes or combinations of electrodes.

In some examples, tissue mapping algorithms 117 may be configured to generate a visual representation of a spatial or temporal temperature distribution of the wall of vessel 120. In some examples, this visual representation may include a two-dimensional or three-dimensional thermal field of the perivascular tissues based on the thermal data at the wall of the vessel. In some examples, tissue mapping algorithms 117 may be configured to classify at least one of the one or more perivascular tissues based on the spatial or temporal temperature distribution of the one or more perivascular tissues. For example, tissue mapping algorithms 117 may be configured to classify spatial areas based on thermal behavior that reflects various thermal properties and/or flow properties of a particular tissue, or a relative position of tissues. In some examples, tissue mapping algorithms 117 may be further configured to use impedance data, in addition to thermal data, to classify spatial areas based on electrical behavior that reflect various electrical properties of a particular tissue or relative position of tissues.

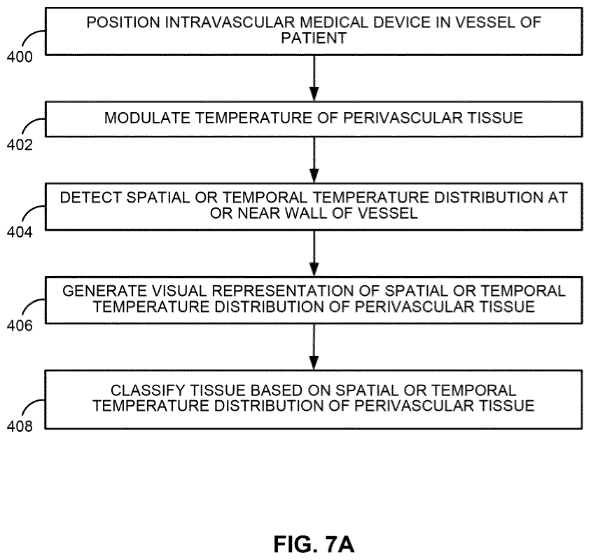

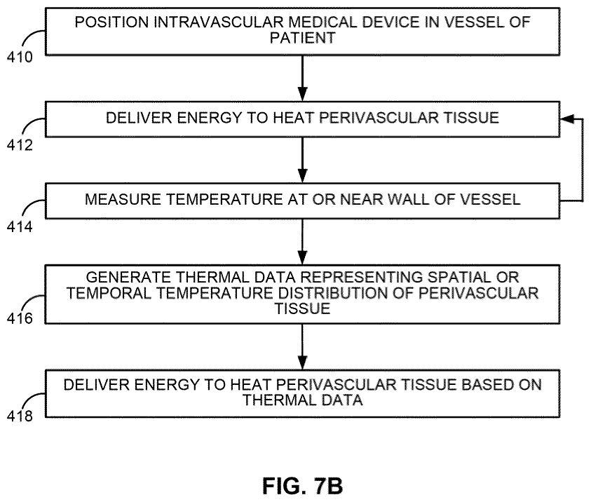

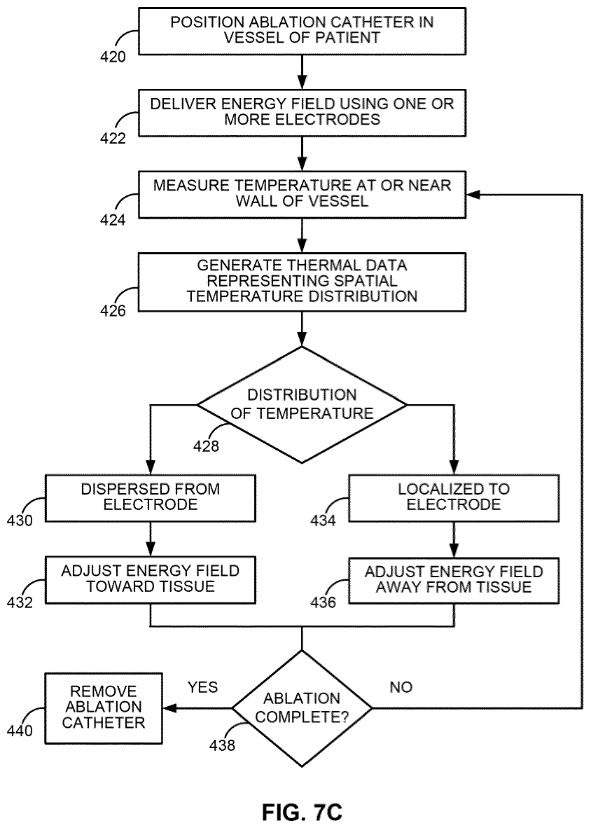

Further operation of control system 104, including control algorithms 116, evaluation/feedback algorithms 118, and tissue mapping algorithms 117, will be described in FIGS. 7A and 7B below. Further, it will be understood that system 100 may include a computing device that includes greater or fewer functions than energy field generator 104. For example, instead of energy field generator 104, system 100 may include a separate computing device configured with evaluation/feedback algorithms 118 for receiving thermal data from intravascular medical device 102 and tissue mapping algorithms 117 for evaluating the thermal data, generating a visual representation of the thermal data, and/or classifying one or more tissues based on the thermal, and optionally impedance, data.

Intravascular medical devices described herein may include any of a variety of configurations, such as various forms of a structural support member, various mechanisms for contacting temperature sensors to a wall of a vessel, or various combinations with or without electrodes. FIGS. 3A-3D illustrate various configurations of intravascular medical devices; however, it will be understood that features of each of the intravascular medical device of FIGS. 3A-3D may be added, removed, or combined in other configurations. The intravascular medical devices described in FIGS. 3A-3D may be used for intravascular medical device 102 of system 100 of FIG. 1, or may be used with other systems.

FIG. 3A is an expanded side view conceptual illustration of a distal portion of an example intravascular medical device 200. Intravascular medical device 200 includes a support structure 204 coupled to a shaft 202, such as shaft 108 of intravascular medical device 102 of FIG. 1. Support structure 204 defines a longitudinal axis 203 and is configured to be positioned within a vessel 210 of a patient. Support structure 204 may be configured to switch between a delivery configuration, in which the support structure 204 is straight, to a deployed configuration as shown, in which support structure 204 is spiraled. In the deployed configuration, support structure 204 may define a perimeter that extends along an inner surface of vessel 210, such that elements at an outer surface of support structure 204 may contact the inner surface of vessel 210. Support structure 204 may be configured to switch between the delivery configuration and the deployed configuration using any of a number of mechanisms, such as actuation of a shape memory member within support structure 204 or actuation of a pull or push wire within support structure 204.

Intravascular medical device 200 includes a plurality of electrodes 208 arranged around the perimeter defined by support structure 204 and a plurality of temperature sensors 206 arranged around the perimeter of support structure 204. The plurality of electrodes 208 may be spaced along support structure 204 such that, when deployed, the electrodes may have an axial spacing along and a circumferential spacing around axis 203. Each of the plurality of electrodes is configured to deliver energy to one or more perivascular tissues near the vessel to heat the one or more perivascular tissues. Each of the plurality of temperature sensors 206 is configured to measure a temperature at or near the wall of the vessel. For example, temperature sensors 206 may be configured, once deployed to face away from axis 203 to contact vessel 210. In some examples, temperature sensors 206 may be band temperature sensors configured to extend around support structure 204, such that a particular directionality of temperature sensors 206 may not be required.

In the example of FIG. 3A, intravascular medical device 200 include support structure 204 configured to expand to contact vessel 210, such that temperature sensors 206 may contact vessel 210. However, in other examples, temperature sensors may be configured to extend from a support structure to contact vessel 210. FIG. 3B is an expanded side view conceptual illustration of a distal portion of an example intravascular medical device 220. Intravascular medical device 220 includes a support structure 224 defining a longitudinal axis 223 and configured to be positioned within vessel 210. Support structure 224 includes an elongated body defining an inner lumen. Intravascular medical device 220 includes a plurality of electrodes 228 arranged around a perimeter of support structure 224. Each of the plurality of electrodes 228 is configured to deliver energy to one or more perivascular tissues near vessel 210 to heat the one or more perivascular tissues.

Intravascular medical device 220 also has a plurality of temperature sensors 226 arranged around the perimeter of support structure 224. Each of the plurality of temperature sensors 226 is configured to measure a temperature at or near the wall of vessel 210. To contact the wall of vessel 210, each of the plurality of temperature sensors 226 is configured to extend from an outer perimeter of support structure 224 to contact and/or penetrate vessel 210. For example, temperature sensors 226 may receive a stronger or more accurate temperature measurement external to vessel 210. Each of the plurality of temperature sensors 226 may be mechanically coupled to an actuation assembly 230 configured to extend the plurality of temperature sensors 226 to, into, or through the wall of vessel 210. A variety of actuation assemblies may be used including, but not limited to, a push wire, and the like.

In the examples of FIGS. 3A and 3B, intravascular medical devices 200 and 220 included both temperatures sensors 206, 226 and electrodes 208, 228. However, in other examples, such as will be described in FIGS. 3C and 3D below, intravascular medical devices described herein may include only temperature sensors, or may be part of an assembly that includes a first intravascular medical device having temperature sensors and a second intravascular medical device having electrodes or another energy source.

FIG. 3C is an expanded side view conceptual illustration of a distal portion of an example intravascular medical device 240. Intravascular medical device 240 includes a support structure 244 coupled to shaft 242, defining a longitudinal axis 243, and configured to be positioned within vessel 210. In the example of FIG. 3C, support structure 244 is an expandable mesh structure configured to expand to contact an inner surface of vessel 210. Other designs of support structure 244 may include, but are not limited to, a balloon, and the like. In some examples, support structure 244 may be self-expanding, while in other examples, support structure 244 may be expanded by an actuator, such as a push or pull wire.

Intravascular medical device 240 includes a plurality of temperature sensors 246 arranged around the perimeter of support structure 244. Each of the plurality of temperature sensors 246 is configured to measure a temperature at or near the wall of vessel 210. For example, the plurality of temperature sensors 246 may contact an inner surface of the wall of vessel 210 when support structure 244 is in a deployed or expanded configuration. In the example of FIG. 3C, intravascular medical device 240 may not include a plurality of electrodes to deliver energy to one or more perivascular tissues near vessel 210 to heat the one or more perivascular tissues. Instead, a separate energy source may be configured to modulate a temperature of the one or more perivascular tissues. As one example, an ultrasound generator or RF electrode device may be positioned in a lumen of support structure 244 and configured to deliver mechanical or electromagnetic energy, respectively, to the perivascular tissues. As another example, a flow of blood or other fluid to the perivascular tissues may be restricted to reduce cooling to the perivascular tissue. In any case, the plurality of temperature sensors 246 may be configured to measure the temperature at or near the wall of vessel 210.

In some examples, intravascular medical device 240 may be part of an intravascular medical device assembly that includes a second intravascular medical device positioned in an adjacent vessel. FIG. 3D is an expanded side view conceptual illustration of a distal portion of an example intravascular medical device assembly 260. Intravascular medical device assembly 260 includes a first intravascular medical device 262 configured to deliver energy to perivascular tissues near a first vessel 264 to heat the perivascular tissues and a second intravascular medical device 240 configured to detect temperature variations in the perivascular tissues.

Intravascular medical device 262 includes support structure 204 coupled to shaft 202, defining longitudinal axis 203, and configured to be positioned within vessel 264. Vessel 264 may be adjacent to vessel 210 and may be separated from vessel 210 by a distance 266. In some examples, distance 266 may be less than about 4 centimeters. Intravascular medical device 262 includes a plurality of electrodes 208 arranged around a perimeter of support structure 204. Each of the plurality of electrodes 208 is configured to deliver energy to one or more perivascular tissues near vessel 264 to heat the one or more perivascular tissues. Intravascular medical device 240 includes a plurality of temperature sensors 246 arranged around the perimeter of support structure 244. Each of the plurality of temperature sensors 246 is configured to measure a temperature at or near the wall of vessel 210. The temperature at or near the wall of vessel 210 may be influenced by a change in temperature of the perivascular tissue induced by the plurality of electrodes 208 of intravascular medical device 262.

In the example of FIG. 3D, intravascular medical device 240 and intravascular medical device 262 are configured to deliver energy to perivascular tissues near first vessel 264 to heat the perivascular tissues and detect temperature variations in the perivascular tissues, respectively. However, in other examples, intravascular medical devices positioned in different vessels may each be configured to both modulate a temperature of one or more perivascular tissues near a respective vessel detect a spatial or temporal distribution of temperatures at or near a wall of the respective vessel. For example, a first intravascular medical device, such as intravascular medical device 200 of FIG. 3A, may be positioned in a first vessel, while a second intravascular medical device, such as intravascular medical device 200 of FIG. 3A, may be positioned in a second vessel. In a first iteration, the first intravascular medical device may modulate a temperature of perivascular tissues, such as tissues between the first and second vessels, and the second intravascular medical device may detect a spatial or temporal distribution of temperatures at or near the wall of the second vessel. In a second iteration, the second intravascular medical device may modulate a temperature of the perivascular tissues, and the first intravascular medical device may detect a spatial or temperature distribution of temperatures at or near the wall of the first vessel. The thermal data generated from the first and second intravascular medical devices may represent both the spatial or temporal temperature distribution of the temperatures at or near the wall of the first vessel and the spatial or temporal temperature distribution of the temperatures at or near the wall of the second vessel, thereby providing a more complete representation of the perivascular tissue.

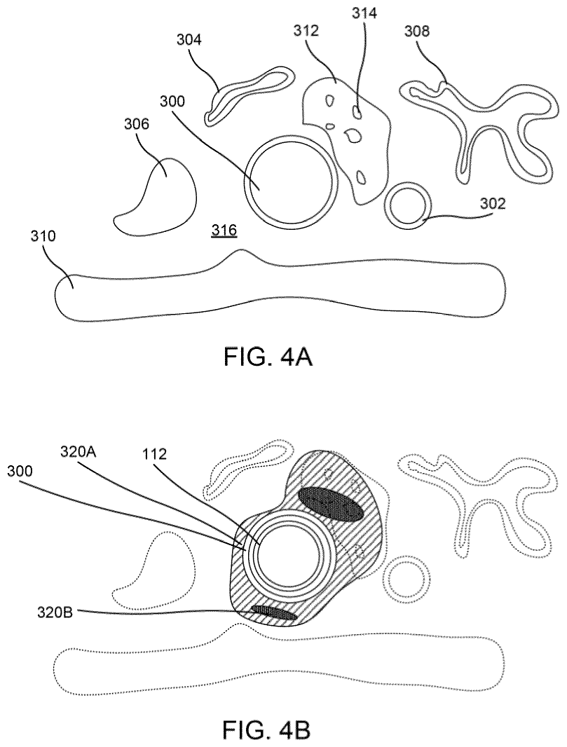

Intravascular medical devices described herein may be used as part of a tissue imaging system to determine a relative location of various tissues within a volume of perivascular tissue. FIG. 4A is a cross-sectional illustration of perivascular tissue near a renal artery 300. The perivascular tissue includes various tissues, such as a secondary renal artery 302, a renal vein 304, a lymph node 306, a ureter 308, a muscle 310 (e.g., psoas), and perivascular fat 312 containing one or more renal nerves 314, and perirenal fat 316. The various tissues in a volume of perivascular tissue may have a different configuration (e.g., presence and/or location) depending on an axial location of renal artery 300. For example, lymph node 136 may be more likely to be present in a volume of perivascular tissue near a proximal portion of renal artery 300, while a renal nerve 314 may be more likely to be present in a volume of perivascular tissue near a distal portion of renal artery 300. The various tissues in a particular volume of perivascular tissue may change from person to person. For example, in a particular cross-section taken at a particular axial location of renal artery 300, a presence or arrangement of the various tissues may differ.

The various tissues in a volume of perivascular tissue may have different electrical, thermal, and flow properties, such as electrical conductivity, thermal conductivity, impedance, heat capacity, fluid flow rate, fluid flow volume, and fluid flow variation. These various properties may affect an amount of heat generated from a particular energy field in the particular tissue and/or an amount of heat removed from fluid flowing through or adjacent to the particular tissue. For example, a particular tissue may produce heat at a rate in response to an energy field and receive and transfer heat from and to adjacent tissues according to various thermal and flow properties of the tissue, and may produce an impedance in response to a current according to various electrical properties of the tissue. The temperature, and optionally impedance, response of the various tissues as a function of space and/or time may provide insight as to an identity and/or relative location of particular tissues in the volume of perivascular tissue.

In some instances, various tissues may have different heat conductivity and capacity properties based on a composition and/or alignment of cells of the tissue. For example, some of the tissues, such as perivascular fat 312 and perirenal fat 316, may be thermal tissues having thermal properties conducive to generating and maintaining heat, while other tissues, such as bone, may be nonthermal tissues having thermal properties that are not conducive to generating and maintaining heat. As one example, some thermal tissues, such as perivascular fat 312, may have a relatively high heat capacity, and therefore a relatively high temperature, while other tissues, such as muscle 310, may have a relatively low heat capacity, and therefore relatively low temperature.

In some instances, various tissues may have different heat removal properties based on a presence, amount, or nature of fluid flowing through a particular tissue or through adjacent tissues. For example, some of the tissues, such as secondary renal artery 302, renal vein 304, lymph node 306, and ureter 308, may be convective tissues having flow of a fluid, while other tissues, such as muscle 310 and perivascular fat 312, may be non-convective tissues that do not have flow. Fluid flowing through convective tissues may remove heat from the convective tissues and other tissues adjacent to the convective tissues. Convective tissues may also have flow properties that differ based on a volume of flow and/or a continuity of flow. As one example, some convective tissues, such as secondary renal artery 302, may have continuous flow of a fluid, and therefore relatively consistent heat removal, while other convective tissues, such as renal vein 304, lymph node 306, or ureter 308, may have discontinuous flow of a fluid, and therefore relatively inconsistent heat removal. As another example, some of these tissues, such as secondary renal artery 302 and renal vein 304, may have relatively high flow rates, and therefore relatively high heat removal, while other tissues, such as lymph node 306 and ureter 308, may have relatively low flow rates, and therefore relatively low heat removal.

FIG. 4B is a cross-sectional illustration of the perivascular tissue of FIG. 3A that includes a thermal field 320 generated from an intravascular medical device positioned in renal artery 3000 of FIG. 3A. In the example of FIG. 3B, thermal field 320 is generated by applying an even amount of energy around thermal modulation and detection element 112 to heat the perivascular tissues. As shown in the FIG. 3B, thermal field 320 includes areas of relatively low temperature increase 320A and regions of relatively high temperature increase 320B. A shape of thermal field 320 may be influenced by the thermal and flow properties of the various tissues in the perivascular tissue and a distance of the various tissues from thermal modulation and detection element 112. For example, fluids flowing through convective tissues such as secondary renal artery 302, renal vein 304, lymph node 306, and ureter 308 may remove a portion of the heat generated from the energy field, resulting in relatively low temperature increases in convective tissues or tissues near convective tissues. In contrast, non-convective tissues and/or thermal tissues such as muscle 310, perivascular fat 312, and perirenal fat 316 may generate and store a relatively large amount of heat, resulting in relatively high temperature increases in non-convective tissues and/or tissues having relatively high thermal conductivities and/or heat capacities. As a result, areas of relatively high temperature increase 320B may be near these non-convective and/or thermal tissues.

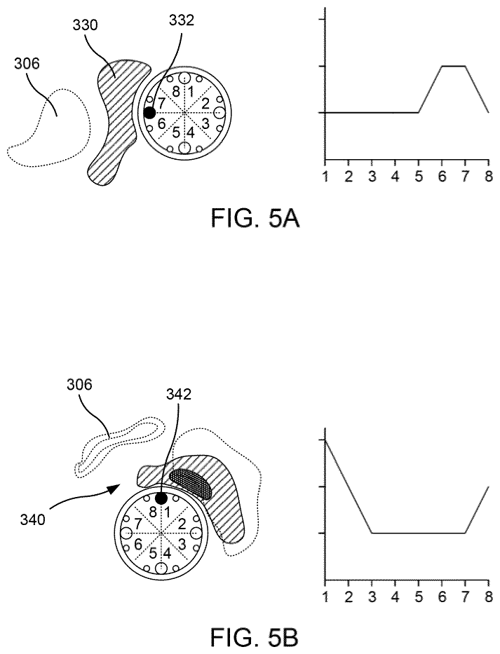

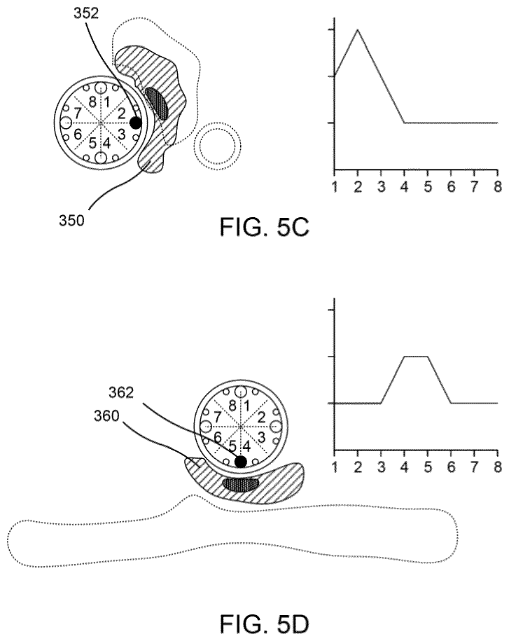

Intravascular medical devices described herein may detect a thermal field in perivascular tissues generated by an energy source. To detect the thermal field in the perivascular tissues, intravascular medical devices may detect and output a spatial or temporal distribution of temperatures at or near a wall of the vessel. To generate the thermal field, the energy source may selectively heat certain portions of the perivascular tissue and detect a distribution of temperatures that result from the selective heating. [0071] FIG. 5A-5D are illustrations of thermal fields generated by an intravascular medical device from various electrodes positioned along a vessel at various circumferential locations. FIGS. SA-5D will be described with respect to the perivascular tissue layout described in FIGS. 4A-4B.

In FIG. 5A, an electrode 332 delivers energy to the perivascular tissue at an 9:00 o'clock position to heat the perivascular tissue adjacent to electrode 332. At least a portion of this heat is removed by lymph node 306, which is a convective tissue that include lymph flowing at a relatively low and discontinuous flow rate. As a result, electrode 332 produces a thermal field 330 that decreases in temperature based on a distance from electrode 332 and a proximity to lymph node 306. As shown in the corresponding graph, thermal field 330 causes temperature sensors located at the 6 and 7 circumferential positions of artery 300 to show a relatively moderate temperature and all other temperature sensors to show a relatively low temperature.

In FIG. 5B, an electrode 342 delivers energy to the perivascular tissue at a 12:00 o'clock position to heat the perivascular tissue adjacent to electrode 342. At least a portion of this heat is removed by renal vein 304, which is a convective tissue that includes blood flowing at a relatively high and discontinuous flow rate. Perivascular fat 312, which is a thermal tissue having a relatively high heat capacity and/or thermal conductivity, may heat to a higher temperature than surrounding tissues that have lower thermal properties or are closer in proximity to renal vein 304. As a result, electrode 342 produces a thermal field 340 that decreases in temperature based on a distance from electrode 342 and a proximity to renal vein 304 and increases in temperature based on a proximity to perivascular fat 312. As shown in the corresponding graph, thermal field 340 causes a temperature sensor located at the 1 circumferential positions of artery 300 to show a relatively high temperature, temperature sensors located at the 2 and 8 circumferential positions of artery 300 to show a relatively moderate temperature, and all other temperature sensors to show a relatively low temperature.

In FIG. 5C, an electrode 352 delivers energy to the perivascular tissue at a 3:00 o'clock position to heat the perivascular tissue adjacent to electrode 352. At least a portion of this heat is removed by secondary renal artery 302, which is a convective tissue that includes blood flowing at a relatively high and continuous flow rate. Perivascular fat 312, which is a thermal tissue having a relatively high heat capacity and/or thermal conductivity, may heat to a higher temperature than surrounding tissues that have lower thermal properties or are closer in proximity to renal artery 302. As a result, electrode 352 produces a thermal field 350 that decreases in temperature based on a distance from electrode 352 and a proximity to renal artery 302 and increases in temperature based on a proximity to perivascular fat 312. As shown in the corresponding graph, thermal field 350 causes a temperature sensor located at the 2 circumferential positions of artery 300 to show a relatively high temperature, temperature sensors located at the 1 and 3 circumferential positions of artery 300 to show a relatively moderate temperature, and all other temperature sensors to show a relatively low temperature.

In FIG. 5D, an electrode 362 delivers energy to the perivascular tissue at a 6:00 o'clock position to heat the perivascular tissue adjacent to electrode 362. Muscle 310, which is a thermal tissue having a relatively moderate heat capacity and/or thermal conductivity, may heat to a higher temperature than surrounding tissues that have lower thermal properties. As a result, electrode 362 produces a thermal field 360 that decreases in temperature based on a distance from electrode 362 and increases in temperature based on a proximity to muscle 310. As shown in the corresponding graph, thermal field 360 causes temperature sensors located at the 4 and 5 circumferential positions of artery 300 to show a relatively moderate temperature, and all other temperature sensors to show a relatively low temperature.

Thermal data detected from thermal fields 330, 340, 350, 360 in FIGS. 5A-5D may represent a spatial distribution of temperatures at or near a wall of artery 300. In some examples, thermal data may further represent a temporal distribution of temperatures at or near a wall of artery 300. A temporal distribution may include temperature measurements over a period of time, with or without energy delivery to the perivascular tissue. As one example, electrodes 342, 342, 352, 362 may deliver energy to heat the perivascular tissue and subsequently refrain from delivering energy to cool the perivascular tissue. In response, different tissues within the perivascular tissue may heat up or cool down at different rates based on different thermal properties, such as heat capacity or thermal conductivity. Referring to FIG. 5B, electrode 342 may deliver energy to the adjacent perivascular tissue, such as continuously or in a pulse, and the temperature sensors may measure a temperature of the perivascular tissue as the perivascular tissue heats up or cools down to identify a relatively high rate of heating of perivascular fat 312 and/or a relatively high rate of cooling of tissues near renal vein 304. As another example, convective tissues may remove heat from the perivascular tissue based on different degrees of continuity, such as relatively continuous flow for arteries or relatively discontinuous flow for ureters, which may be detected by monitoring temperature fluctuations of the perivascular tissue over a period of time. Referring to FIG. 5C, temperature sensors may measure a temperature of the perivascular tissue over a period of time to identify a relatively constant rate of cooling of renal artery 302. The temperature sensors corresponding to various circumferential positions in artery 300 may measure the temperature during this heating up or cooling down and output thermal data that further represents a spatial distribution of rates or patterns of heating or cooling at or near a wall of artery 300. These rates or patterns of heating and/or cooling may be used to differentiate particular tissues.

In addition to selectively heating perivascular tissues using electrodes 332, 342, 352, 362, the temperatures of the one or more tissues may be modulated by modulating flow of fluid to various convective tissues. Referring to FIG. 5B, flow of blood through renal vein 306 may be restricted, such that less heat may be removed from the perivascular tissues adjacent to electrode 342, resulting in a thermal field that has a higher temperature at the 8 circumferential position. This temperature increase may indicate that renal vein 306 is relatively close to the 8 circumferential position.

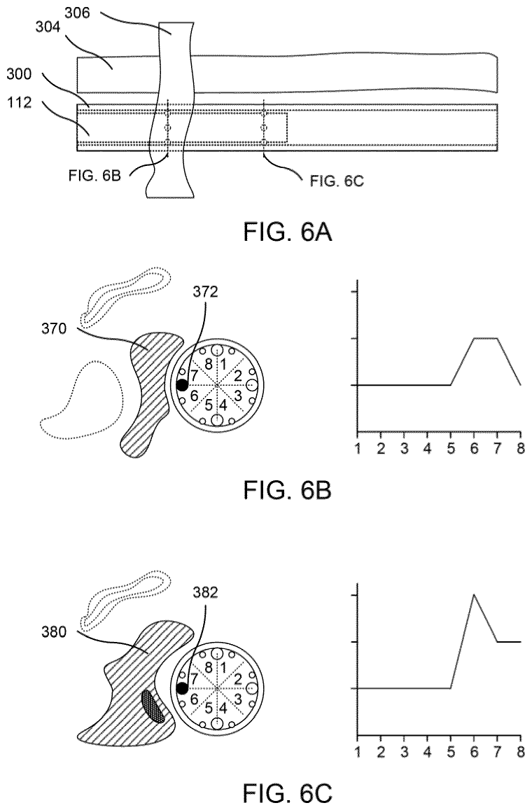

FIG. 6A is a cross-sectional illustration of perivascular tissue near a renal artery 300, while FIGS. 6B-6C are illustrations of thermal fields generated by an intravascular medical device from various electrodes positioned along renal artery 300 at various axial locations. FIGS. 6B and 6C will be described with respect to the perivascular tissue layout described in FIGS. 4A-4B and 6A.

Referring to FIG. 6A, the perivascular tissue includes renal vein 304 extending generally parallel to artery 300 and lymph node 306 extending generally perpendicular to artery 300, as is generally the case in anatomy of a patient. Thermal modulation and detection element 112 is positioned within artery 300, such that a first set of temperature sensors is proximate to both renal vein 304 and lymph node 306 and a second set of temperature sensors is distal to lymph node 306.

In FIG. 6B, an electrode 372 at a first axial position delivers energy to the perivascular tissue at a 9:00 o'clock position to heat the perivascular tissue adjacent to electrode 372. At least a portion of this heat is removed by lymph node 306, which is a convective tissue that include lymph flowing at a relatively low and discontinuous flow rate, and by renal vein 304, which is also a convective tissue that includes blood flowing at a relatively high and discontinuous flow rate. As a result, electrode 372 produces a thermal field 370 that decreases in temperature based on a distance from electrode 372 and a proximity to lymph node 306 and renal vein 304. As shown in the corresponding graph, thermal field 370 causes temperature sensors located at the 6 and 7 circumferential positions of artery 300 to show a relatively moderate temperature and all other temperature sensors to show a relatively low temperature.

In FIG. 6C, an electrode 382 at a second, more distal axial position delivers energy to the perivascular tissue at a 9:00 position to heat the perivascular tissue adjacent to electrode 382. At least a portion of this heat is removed by renal vein 304, which is also a convective tissue that includes blood flowing at a relatively high and discontinuous flow rate, but not by lymph node 306. As a result, electrode 382 produces a thermal field 380 that decreases in temperature based on a distance from electrode 382 and a proximity to renal vein 304. As shown in the corresponding graph, thermal field 380 causes temperature sensor 6 located at the 6 circumferential position of artery 300 to show a relatively high temperature, temperature sensors located at the 7 and 8 circumferential positions of artery 300 to show a relatively moderate temperature, and all other temperature sensors to show a relatively low temperature.

Intravascular medical devices configured in accordance with at least some embodiments of the present technology can be well suited (e.g., with respect to sizing, flexibility, operational characteristics, and/or other attributes) for mapping perivascular tissues around renal arteries and, optionally, performing renal neuromodulation in human patients. Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically-induced, thermally-induced, or induced in another suitable manner or combination of manners at one or more suitable treatment locations during a treatment procedure. The treatment location can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery.

Renal neuromodulation can include an electrode-based treatment modality alone or in combination with another treatment modality. Electrode-based treatment can include delivering electricity and/or another form of energy to tissue at or near a treatment location to stimulate and/or heat the tissue in a manner that modulates neural function. For example, sufficiently stimulating and/or heating at least a portion of a sympathetic renal nerve can slow or potentially block conduction of neural signals to produce a prolonged or permanent reduction in renal sympathetic activity. A variety of suitable types of energy can be used to stimulate and/or heat tissue at or near a treatment location. For example, neuromodulation in accordance with embodiments of the present technology can include delivering RF energy, pulsed electrical energy, microwave energy, and/or another suitable type of energy. An electrode used to deliver this energy can be used alone or with other electrodes in a multi-electrode array.

Heating effects of electrode-based treatment can include ablation and/or non-ablative alteration or damage (e.g., via sustained heating and/or resistive heating). For example, a treatment procedure can include raising the temperature of target neural fibers to a target temperature above a first threshold to achieve non-ablative alteration, or above a second, higher threshold to achieve ablation. The target temperature can be higher than about body temperature (e.g., about 37° C.) but less than about 45° C. for non-ablative alteration, and the target temperature can be higher than about 45° C. for ablation. Heating tissue to a temperature between about body temperature and about 45° C. can induce non-ablative alteration, for example, via moderate heating of target neural fibers or of luminal structures that perfuse the target neural fibers. In cases where luminal structures are affected, the target neural fibers can be denied perfusion resulting in necrosis of the neural tissue. Heating tissue to a target temperature higher than about 45° C. (e.g., higher than about 60° C.) can induce ablation, for example, via substantial heating of target neural fibers or of luminal structures that perfuse the target fibers. In some patients, it can be desirable to heat tissue to temperatures that are sufficient to ablate the target neural fibers or the luminal structures, but that are less than about 90° C. (e.g., less than about 85° C., less than about 80° C., or less than about 75° C.).

In some examples, tissues intended for treatment, such as renal nerves in renal neuromodulation described above, may be mapped prior to treatment to determine a relative location of the tissues. For example, prior to performing an ablation procedure, perivascular tissue of the renal artery may be heated and mapped to determine a relative position of convective tissues that may draw heat away from the perivascular tissues or thermal tissues that may include tissues of interest, such as renal nerves. FIG. 7A is a flowchart of an example method for generating and detecting a thermal field in perivascular tissue in accordance with some examples of the present disclosure.

The method may include positioning intravascular medical device 102, or any of intravascular medical devices 200, 220, 240, or 262, in vessel 120 of a patient (400). For example, for a renal artery mapping procedure, positioning intravascular medical device 102 may include inserting intravascular medical device 102 into a femoral artery of the patient and navigating intravascular medical device 102 through the vasculature of the patient to a renal artery, such that thermal modulation and detection element 112 is positioned near a desired mapping location. The position of thermal modulation and detection element 112 may be confirmed through imaging. In examples in which intravascular medical device 102 includes an expandable support structure or extendable temperature sensors, intravascular medical device 102 may be expanded and/or temperature sensors 124 may be extended, such that temperature sensors 124 may contact an inner wall of vessel 120. In examples in which intravascular medical device 102 is part of an intravascular medical assembly, such as described in FIG. 3D, a first intravascular medical device 240 for detecting a thermal field in the perivascular tissue may be positioned in a first vessel 210 and a second intravascular medical device 262 for generating an energy field that produces the thermal field in the perivascular tissue may be positioned in a second vessel 264. In some examples, distance 266 between first vessel 210 and second vessel 264 may be less than about four centimeters.

The method includes modulating a temperature of one or more perivascular tissues near vessel 120 of the patient (402). Modulating the temperature of the one or more perivascular tissues comprises delivering energy to or removing energy from the one or more perivascular tissues. This energy may be delivered to or removed from vessel 120 in which intravascular medical device 102 is positioned, from an adjacent vessel to vessel 120, such as a vessel that includes a second intravascular medical device for generating an energy field, or structures within the perivascular tissue itself, such as convective tissues. A variety of different types of energy may be used including, but not limited to, radiofrequency energy, ultrasound energy, conductive thermal energy, convective thermal energy, radiative thermal energy, or any other energy configured to generate a temperature response from the perivascular tissues resulting in a thermal field.

In some examples, modulating the temperature of the one or more perivascular tissues includes controlling an energy source to deliver energy to the one or more perivascular tissues to heat the one or more perivascular tissues. The energy field delivered to the perivascular tissues may have a distribution based on properties of the energy delivered, properties of the energy source, a location of the energy source, and properties of the perivascular tissues. In the example intravascular medical device 102 of FIGS. 2A-2B, the energy source includes the plurality of focal energy sources 126 arranged around the perimeter of support structure 122. The energy is delivered to the one or more perivascular tissues using the plurality of focal energy sources 126, such as in a particular sequence according to an axial or circumferential position, at a particular strength, or for a particular duration. In examples in which intravascular medical device 102 is part of an intravascular medical assembly, such as described in FIG. 3D, modulating the temperature of the one or more perivascular tissues includes delivering, from second intravascular medical device 262 positioned in second vessel 264, energy to the one or more perivascular tissues.

In some examples, modulating the temperature of the one or more perivascular tissues includes modulating a flow rate of blood to the one or more perivascular tissues. For example, as illustrated in FIG. 4A, perivascular tissue may include blood vessels, such as arteries or veins, that may flow blood and remove heat from the perivascular tissues. Modulating the flow rate of blood, such as by restricting the flow of blood for a period of time, may reduce a cooling effect of the blood through the perivascular tissue, resulting in higher tissue temperatures near the blood vessels.

Modulating the temperature of the perivascular tissues results in a thermal field in the perivascular tissues. For example, prior to modulating the temperature, the perivascular tissues may have an initial distribution of temperatures based on a relatively steady state that may include minor temperature fluctuations from periodic changes in fluid flow to the perivascular tissues. After modulating the temperature, the perivascular tissues may have a different distribution of temperatures that depends on properties of the tissues. As such, the thermal field generated by the energy field may represent a spatial distribution of temperatures. The spatial distribution of temperatures may include an axial component along vessel 120, a circumferential component around vessel 120, and a radial component away from vessel 120. The spatial distribution of temperatures in the thermal field may correspond to a spatial distribution of temperatures at the wall of vessel 120. For example, perivascular tissue at a particular circumferential, axial, or radial location within the perivascular tissue may exert a greater amount of influence (e.g., transfer of a greater amount of heat) on the temperature of a proximate portion of the wall of vessel 120 perivascular tissues that are further away.

The method includes detecting a spatial or temporal distribution of temperatures at or near a wall of vessel 120 (404). In some examples, detecting the spatial distribution of temperatures includes measuring, by each of the plurality of temperature sensors 124, a temperature at or near the wall of vessel 120 and generating, based on one or more temperature measurements of the temperature at or near the wall of vessel 120, thermal data representing the spatial temperature distribution of the temperatures. For example, each temperature measurement may represent the temperature of the wall of vessel 120 at a respective axial and circumferential position on support structure 122. As a result, the thermal data includes the spatial temperature distribution of the temperature of the wall of vessel 120 at the respective axial and circumferential position on support structure 122.

In some examples, detecting the spatial distribution of temperatures includes measuring, by each of the plurality of temperature sensors 124, a temperature at or near the wall of vessel 120 and generating, based on one or more temperature measurements of the temperature at or near the wall of vessel 120, thermal data representing the spatial temperature distribution of the temperatures. For example, each temperature measurement may represent the temperature of the wall of vessel 120 at a respective axial and circumferential position on support structure 122. As a result, the thermal data includes the spatial temperature distribution of the temperature of the wall of vessel 120 at the respective axial and circumferential position on support structure 122.

In some examples, the method includes delivering current to the perivascular tissues and detecting a spatial distribution of impedances of the perivascular tissues. In some examples, delivering the current and detecting the spatial distribution of impedances includes delivering, by one or more of the plurality of electrodes, a current to the wall of vessel 120, measuring, by one or more of the plurality of focal energy sources 126, an impedance at the wall of vessel 120, and generating, based on one or more impedance measurements of the impedance at or near the wall of vessel 120, impedance data representing the spatial impedance distribution of the impedances.

In some examples, the method may include generating, based on the thermal data, a visual representation of the spatial or temporal temperature distribution of the temperatures at or near the wall of vessel 120 (406). For example, a tissue mapping system may receive the thermal data and generate image data that represents a two-dimensional or three-dimensional representation of the temperature distribution at various axial and/or circumferential positions on the inner surface of vessel 120, such as a heat map. In some examples, the tissue mapping system may generate image data that represents a visual representation of a thermal field of the perivascular tissue based on the thermal data. For example, the tissue mapping system may use thermal data captured for energy fields produced by different focal energy sources 126 or combinations of focal energy sources 126 and generate image data that represents a two-dimensional or three-dimensional representation of the temperature distribution at various axial, circumferential, and/or radial positions in the perivascular tissue relative to vessel 120.

In some examples, the method may include classifying at least one of the one or more perivascular tissues based on the spatial or temporal temperature distribution of at the wall of vessel 120 (408). For example, different perivascular tissues may have different thermal, electrical, and flow properties, and may be located at different distances from intravascular medical device 120. The spatial or temporal temperature distribution of the perivascular tissue may reflect these different properties and/or different relative distances, such that various tissues within the perivascular tissue may be differentiated and identified.

In some examples, the tissues may be classified based on different thermal or flow properties. For example, a tissue mapping system may receive thermal data that includes temperatures at various axial and circumferential locations in vessel 120 produced by a particular energy field. The tissue mapping system may determine one or more thermal or flow properties for tissues from the temperatures. For example, the tissue mapping system may identify a spatial region according to thermal behavior in response to the energy field, such as a magnitude of the temperature or rate of the temperature increase or decrease. The temperature behavior may reflect various thermal properties of a thermal tissue, various flow properties of a convective tissue, and/or various effects of differences in thermal or flow properties according to a distance of the spatial region from vessel 120. This temperature behavior may act as a thermal signature corresponding to particular tissue types. The tissue mapping system may classify the spatial region of tissue as a particular tissue type based on the thermal properties or flow properties, such as by matching the temperature behavior to known thermal signatures corresponding to particular tissue types. The known thermal signatures may be determined, such as through experimental data, by correlating particular thermal behavior with resulting physiological responses in tissue function or anatomical changes in tissue structure.

In some examples, the tissues may be classified based on different electrical properties. For example, in addition to receiving thermal data representing a spatial or temporal temperature distribution of the perivascular tissues, a tissue mapping system may receive impedance data representing a spatial impedance distribution of the one or more perivascular tissues. The tissue mapping system may receive both impedance data that includes an impedance of a volume of tissue produced by a particular current and thermal data that includes a temperature or change in temperature of the volume of tissue produced by a particular energy field, determine one or more electrical properties from the impedance data and one or more thermal properties based on the thermal data, and classify the volume of tissue as a particular tissue type based on both the one or more thermal properties and the one or more electrical properties.

In some examples, tissues intended for heat-based ablation, such as renal neuromodulation described above, may be heated and mapped prior to or during ablation to more accurately or efficiently generate an energy field to ablate the tissues. For example, prior to or during a heat ablation procedure, perivascular tissue of the renal artery may be heated to determine tissues that may better respond to heat treatment, such as volumes away from convective tissues or tissues likely to include renal nerves. In response to this temperature feedback, the energy field used to ablate the tissues may be reconfigured to target these tissues. FIG. 7B is a flowchart of an example method for using an ablation catheter to generate and detect a thermal field for controlling an energy field in accordance with some examples of the present disclosure.

The method may include positioning intravascular medical device 102, or any of intravascular medical devices 200, 220, 240, or 262, in vessel 120 of a patient (410), such as described in step 400 of FIG. 7A above. In the example of FIG. 7B, intravascular medical device 102 may be an ablation catheter configured to ablate perivascular tissues using thermal modulation and detection element 112. In other examples, such as illustrated in FIG. 3D, a first intravascular medical device 262 may be an ablation catheter configured to deliver energy to perivascular tissues near first vessel 264 to heat the perivascular tissues, and a second intravascular medical device 240 may be an imaging catheter configured to detect temperature variations in the perivascular tissues.

The method includes delivering energy to the one or more perivascular tissues to heat the one or more perivascular tissues (412). In the example intravascular medical device 102 of FIGS. 2A-2B, the energy is delivered to the one or more perivascular tissues using the plurality of focal energy sources 126, such as in a particular sequence according to an axial or circumferential position, at a particular strength, or for a particular duration, and may be controlled by energy field generator 104 of FIG. 1.

In some examples, the energy delivered to the one or more perivascular tissues may heat the one or more perivascular tissues to a temperature below an ablation temperature of the perivascular tissues. For example, the perivascular tissues may begin to die at temperatures at or above about 60° C. Prior to ablation, one or more thermal fields may be generated in the perivascular tissues and subsequently detected to determine a relative location of perivascular tissues. Energy field generator 104 may operate in an imaging mode to control the plurality of focal energy sources 126 to maintain a temperature of the one or more perivascular tissues below an ablation temperature of the one or more perivascular tissues. For example, energy field generator 104, via the plurality of focal energy sources 126, may maintain the temperatures of the one or more perivascular tissues below about 60° C.

In some examples, the energy delivered to the one or more perivascular tissues may heat the one or more perivascular tissues to a temperature at or above an ablation temperature of the perivascular tissues. Rather than generate and detect thermal fields prior to ablation, one or more thermal fields may be generated in the perivascular tissues and subsequently detected during an ablation procedure to determine a relative location of perivascular tissues. Energy field generator 104 may operate in an ablation mode to control the plurality of focal energy sources 126 to maintain a temperature of the one or more perivascular tissues at or above an ablation temperature of the one or more perivascular tissues for a period of time sufficient to ablate at least a portion of the perivascular tissues. For example, energy field generator 104, via the plurality of focal energy sources 126, may maintain the temperatures of the one or more perivascular tissues at or above about 60° C.

The method includes measuring, by each of the plurality of temperature sensors 124, a temperature at or near the wall of vessel 120 (414). For example, during an ablation procedure, the plurality of temperature sensors 124 may measure the temperature at or near the wall of vessel 120 for a period of time, such as during heat up of the perivascular tissue, ablation at least a portion of the perivascular tissue, and cool down of the perivascular tissue.

The method includes generating, based on one or more temperature measurements of the temperature at or near the wall of vessel 120, thermal data representing the spatial or temporal temperature distribution of the temperatures (416). For example, each temperature measurement may represent the temperature of the wall of vessel 120 at a respective axial and circumferential position on support structure 122. As a result, the thermal data includes the spatial or temporal temperature distribution of the temperature of the wall of vessel 120 at the respective axial and circumferential position on support structure 122.

In some examples, the thermal data may be used to generate a visual representation of the perivascular tissue, such as described in step 406 of FIG. 7A, or classify the perivascular tissue, such as described in step 408 of FIG. 7A. However, in other examples, such as illustrated in FIG. 7B, the method may involve further modifying the energy delivered to the perivascular tissue based on the thermal data (418). The spatial or temporal temperature distribution of the perivascular tissue may indicate tissues of interest (e.g., renal nerves), tissues that may include tissues of interest (e.g., perivascular fat that includes renal nerves), tissues that may be particularly responsive to application of an energy field (e.g., thermal tissues or tissues away from convective tissues), or tissues that have undergone ablation (e.g., tissues that have changed properties due to death of the tissue). The spatial or temporal temperature distribution may be used as feedback to adjust a position of the plurality of focal energy sources 126 and/or adjust an amount of energy delivered from particular focal energy sources 126 of the plurality of focal energy sources 126.

In some examples, the method includes automatically adjusting the energy delivered to the perivascular tissue based on the thermal data. As one example, energy field generator 104 may identify a particular circumferential location of the wall of vessel 120 is associated with a high relative temperature and increase an amount of energy delivered to an electrode 126 near that particular circumferential location. As another example, energy field generator may identify a temperature or change in temperature at a particular circumferential location of the wall of vessel 120 that is typically associated with a temperature and time sufficient to ablate a tissue or a change in material properties resulting from ablation and reduce an amount of energy delivered to an electrode 126 near that particular circumferential location. In some examples, the method includes manually adjusting the energy delivered to the perivascular tissue based on the thermal data. For example, a clinician may look at a visual representation of the spatial or temporal temperature distribution of the perivascular tissue to identify a desired tissue for treatment and adjust a position of the plurality of focal energy sources 126 or an amount of energy to the plurality of focal energy sources 126 to increase the temperature at the desired tissue.

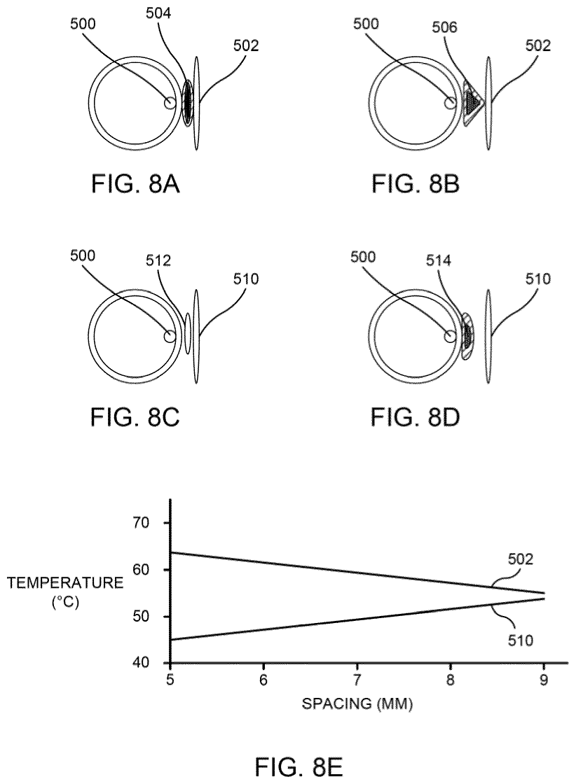

In some examples, operation of an ablation catheter may be modified based on proximity of one or more electrodes to tissues that exhibit thermal behavior indicative of more efficient heating. For example, convective tissues may draw heat away from adjacent tissues, such that delivering energy to these tissues may result in reduced ablation of the tissues. In contrast, thermal tissues may heat to higher temperatures in response to the energy field, such that targeting these tissues may result in increased ablation of the tissues. FIG. 8A-8E are illustrations of simulated thermal fields and a corresponding graph delivered to different tissues at different distances. FIG. 8A illustrates an electrode 500 applying an energy field to a non-convective tissue 502 at a close distance. The resulting thermal field 504 observed at the vessel has a high temperature due to a relative low amount of heat being removed and the close proximity of non-convective tissue 502 to electrode 500. FIG. 8B illustrates electrode 500 applying an energy field to non-convective tissue 502 at a further distance. The resulting thermal field 506 observed at the vessel has a relatively lower temperature and larger distribution due to further proximity of non-convective tissue 502 to electrode 500, and thus reduced concentrating effect. FIG. 8C illustrates electrode 500 applying an energy field to a convective tissue 510 at a close distance. The resulting thermal field 512 observed at the vessel has a very low temperature due a relative high amount of heat being removed compared to non-convective tissue 502 and the close proximity of convective tissue 510 to electrode 500. FIG. 8D illustrates electrode 500 applying an energy field to convective tissue 510 at a further distance. The resulting thermal field 514 observed at the vessel has a relatively higher temperature and larger distribution compared to thermal field 512 due to further proximity of convective tissue 510) to electrode 500, and thus reduced cooling effect. FIG. 8E illustrates a relative temperature measured at a wall of a vessel based on a distance from a non-convective tissue 502 or convective tissue 510. As seen in FIG. 8E, a heating or cooling effect of a respective non-convective or convective tissue may decrease as a proximity to the vessel decreases.

FIG. 7C is a flowchart of an example method for controlling an ablation catheter based on predicted proximity to convective tissue. The method includes positioning an ablation catheter, such as intravascular medical device 102, in vessel 120 of a patient (420). For example, the ablation catheter may be positioned proximate to a known or likely location of renal nerves.

The method includes delivering an energy field to the perivascular tissue using one or more focal energy sources 126 (422). For example, energy field generator 104 may deliver energy to particular electrodes to produce an energy field having axial, circumferential, and radial parameters based on a position of a respective electrode 126 in vessel 120 and/or a magnitude of energy from the respective electrode 126. The method includes measuring a temperature at or near the wall of vessel 120 using the plurality of temperature sensors 124 (424).

The method includes generating thermal data representing a spatial distribution of the temperatures at or near the wall of vessel 120. In some examples, the thermal data may be used to generate a visual representation, such as a thermal field map, of the spatial distribution of the temperatures at or near the wall of vessel 120, such as to guide a clinician in positioning or modifying operation of the ablation catheter. Such a thermal field map may indicate a magnitude of the temperature at various circumferential locations on the wall of vessel 120 or, in some instances, may indicate a magnitude at various radial locations beyond the wall of vessel 120. In other examples, the thermal data may be used to automatically modify operation of the ablation catheter to increase an effectiveness of the energy field in ablating the tissues.

The method includes evaluating, for each electrode, a distribution of a temperature produced by the portion of energy field near the electrode (428). As described in FIGS. 8A-8E above, a thermal field that is localized to the electrode (434) characterized by a relatively low temperature close to an electrode that quickly drops off may be indicative of a convective tissue proximate to the electrode. In contrast, a thermal field that is dispersed from the electrode (430) characterized by a relatively high temperature that extends radially into the tissue may be indicative of a non-convective tissue proximate to the electrode.

In some instances, an indication of a convective tissue may indicate to a clinician that application of an energy field near the convective tissue may be less effective due to removal of heat from the area. In response to the localized distribution of temperature, a clinician or energy field generator 104 may adjust the energy field away from the tissue (436), such as by reducing a magnitude of energy delivered from the proximate electrode or repositioning the ablation catheter. In contrast, in response to the dispersed distribution of temperature, a clinician or energy field generator 104 may adjust the energy field toward the tissue (436), such as by increasing a magnitude of energy delivered from the proximate electrode or repositioning the ablation catheter.

In some instances, an indication of a convective tissue may indicate to a clinician that application of an energy field may target particular tissues for ablation, despite the heat removal effects of the convective tissue. For example, if it is determined, such as for a particular patient or a more generalized group of patients, that a particular tissue is more likely to be present near particular convective tissues, such as between a vein and an artery, application of the energy field near the convective tissues may still effectively ablate the particular tissues due to a higher concentration of renal nerves outweighing any heat removal effects of the convective tissues. In response to a localized distribution of temperature, a clinician or energy field generator 104 may adjust the energy field away toward the tissue (436), such as by increasing a magnitude of energy delivered from the proximate electrode or repositioning the ablation catheter. In this way, knowledge of the configuration of tissues within the perivascular tissue may be used in combination with identifying particular tissues types to target particular tissues.

The method includes continuing to deliver energy to the perivascular tissues for a particular duration and/or to a particular temperature corresponding to a completed ablation (438). Once ablation is complete, the clinician may remove the ablation catheter (440). In this way, systems described herein may more effectively ablate perivascular tissue using temperature feedback.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure. For example, while particular features of the neuromodulation catheters were described as being part of a single device, in other examples, these features can be included on one or more separate devices that can be positioned adjacent to and/or used in tandem with the neuromodulation catheters to perform similar functions to those described herein.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein. The features of various embodiments can be combined to form further embodiments of the invention that may not be explicitly described or illustrated. Each embodiment and each aspect so defined may be combined with any other embodiment or with any other aspect unless clearly indicated to the contrary.

Further, although techniques have been described in which a neuromodulation catheter is positioned at a single location within a single renal artery, in other examples, the neuromodulation catheter may be repositioned to a second treatment site within the single renal artery (e.g., proximal or distal of the first treatment site), in a branch of the single artery, within a different renal vessel on the same side of the patient (e.g., a renal vessel associated with the same kidney of the patient), in a renal vessel on the other side of the patient (e.g., a renal vessel associated with the other kidney of the patient), or any combination thereof. In some examples, thermal data obtained at the first treatment site may be used to inform treatment at the second treatment site. For example, if the second treatment site is relatively close axially to the first treatment site, relative locations of particular tissues or an effect of a particular energy field may be similar at the first and second treatment sites, such that a similar energy field may be applied at the second treatment site as the first treatment site. For instance, a parallel tissue may identified, such as a renal vein, whose orientation is likely to continue from the first treatment site to the second treatment site, in contrast to another tissue, such as a lymph node, that may be more localized. As another example, if thermal data from the first treatment site indicates a relative location of target tissue to an adjacent tissue, such as a nerve to fatty tissue, identification of the adjacent tissue may indicate that application of the energy field toward that tissue may be more effective. At each location where the neuromodulation catheter is positioned, renal neuromodulation may be performed using any of the techniques described herein or any other suitable renal neuromodulation technique, or any combination thereof.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “about” or “approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Aspects and embodiments of the invention may be defined by the following clauses.
Clause 1. An intravascular medical device, comprising:
   a support structure defining a longitudinal axis and configured to be positioned within a vessel of a patient;
   a plurality of focal energy sources arranged around a perimeter of the support structure, wherein each of the plurality of focal energy sources is configured to deliver energy to one or more perivascular tissues near the vessel to heat the one or more perivascular tissues; and
   a plurality of temperature sensors arranged around the perimeter of the support structure, wherein each of the plurality of temperature sensors is configured to measure a temperature at or near a wall of the vessel.
Clause 2. The intravascular medical device of clause 1, wherein each of the plurality of temperature sensors is further configured to contact the wall of the vessel.
Clause 3. The intravascular medical device of clause 2, wherein the support structure is configured to radially expand from a delivery configuration to a deployed position to cause the plurality of temperature sensors to contact the wall of the vessel.
Clause 4. The intravascular medical device of any of clauses 1 to 3, further comprising an actuation assembly configured to extend the plurality of temperature sensors through the wall of the vessel.
Clause 5. The intravascular medical device of any of clauses 1 to 4, wherein at least a portion of the plurality of focal energy sources comprise a plurality of electrodes configured to:
   deliver a current to the wall of the vessel; and
   measure an impedance from the wall of the vessel, wherein the impedance represents an impedance of the one or more perivascular tissues.
Clause 6. The intravascular medical device of any of clauses 1 to 5, wherein each of the plurality of focal energy sources is independently operable.
Clause 7. The intravascular medical device of any of clauses 1 to 6,
   wherein each of the plurality of temperature sensors corresponds to a particular axial and circumferential position on the support structure,
   wherein the intravascular medical device is configured to output a temperature signal that includes a temperature measurement from each of the plurality of temperature sensors, and
   wherein each temperature measurement represents the temperature of the wall of the vessel at the respective axial and circumferential position on the support structure.
Clause 8. The intravascular medical device of any of clauses 1 to 7, wherein the plurality of focal energy sources comprises a plurality of radiofrequency electrodes.
Clause 9. The intravascular medical device of any of clauses 1 to 8, wherein a spacing of adjacent temperature sensors of the plurality of temperature sensors is less than about 10 mm.
Clause 10. The intravascular medical device of any of clauses 1 to 9, wherein an angular spacing of adjacent temperature sensors of the plurality of temperature sensors around the perimeter of the support structure is less than or equal to about 90 degrees.
Clause 11. The intravascular medical device of any of clauses 1 to 10, further comprising an energy field generator communicatively coupled to the plurality of focal energy sources and configured to control the plurality of focal energy sources to deliver the energy to the one or more perivascular tissues.
Clause 12. The intravascular medical device of clause 11, wherein the energy field generator is configured to, in an imaging mode, control the plurality of focal energy sources to heat the one or more perivascular tissues below an ablation temperature of the one or more perivascular tissues.
Clause 13. The intravascular medical device of clause 11 or 12, wherein the energy field generator is further configured to, in an ablation mode, control the plurality of focal energy sources to heat the one or more perivascular tissues above an ablation temperature of the one or more perivascular tissues.
Clause 14. The intravascular medical device of any of clauses 11 to 13, wherein the energy field generator is configured to:
   receive a temperature signal that includes a temperature measurement from each of the plurality of temperature sensors; and
   modify, based on the temperature signal, the energy delivered to at least one focal energy sources of the plurality of focal energy sources.
Clause 15. The intravascular medical device of any of clauses 1 to 14, wherein support structure comprises an ablation catheter.
Clause 16. A tissue mapping system, comprising:
   an intravascular medical device, comprising:
   a support structure defining a longitudinal axis and configured to be positioned within a vessel of a patient; and
   a plurality of temperature sensors arranged around a perimeter of the support structure, wherein the wherein each of the plurality of temperature sensors corresponds to a particular axial and circumferential position on the support structure, and wherein each of the plurality of temperature sensors is configured to measure a temperature at or near the wall of the vessel; and
   a tissue mapping device comprising processing circuitry configured to receive a temperature signal that includes a temperature measurement from each of the plurality of temperature sensors, wherein each temperature measurement represents the temperature of the wall of the vessel at the respective axial and circumferential position on the support structure.
Clause 17. The tissue mapping system of clause 16, wherein the processing circuitry is further configured to generate, based on the one or more temperature measurements, thermal data representing a spatial or temporal temperature distribution of one or more perivascular tissues near the vessel.
Clause 18. The tissue mapping system of clause 17, wherein the thermal data includes a spatial or temporal representation of the temperature of the wall of the vessel at the respective axial and circumferential position on the support structure.
Clause 19. The tissue mapping system of clause 17 or 18, wherein the processing circuitry is configured to classify at least one of the one or more perivascular tissues based on the spatial or temporal temperature distribution of the one or more perivascular tissues.
Clause 20. A method, comprising:
   modulating a temperature of one or more perivascular tissues near a vessel of a patient; and
   detecting, using an intravascular medical device positioned in the vessel, a spatial or temporal distribution of temperatures at or near a wall of the vessel,
   wherein the intravascular medical device comprises a support structure defining a longitudinal axis and a plurality of temperature sensors arranged around a perimeter of the support structure.
Clause 21. The method of clause 20, further comprising positioning the intravascular medical device in the vessel of the patient.
Clause 22. The method of clause 20 or 21, wherein detecting the spatial or temporal distribution of temperatures comprises:
   measuring, by each of the plurality of temperature sensors, a temperature at or near the wall of the vessel; and
   generating, based on one or more temperature measurements of the temperature at or near the wall of the vessel, thermal data representing the spatial or temporal temperature distribution of the temperatures.
Clause 23. The method of clause 22,
   wherein each temperature measurement represents the temperature of the wall of the vessel at a respective axial and circumferential position on the support structure, and
   wherein the thermal data includes the spatial or temporal temperature distribution of the temperature of the wall of the vessel at the respective axial and circumferential position on the support structure.
Clause 24. The method of clause 22 or 23, further comprising generating, based on the thermal data, a visual representation of the spatial or temporal temperature distribution of the temperatures.
Clause 25. The method of any of clauses 22 or 23,
   wherein modulating the temperature of the one or more perivascular tissues comprises delivering a first amount of energy to the one or more perivascular tissues, and
   wherein the method further comprises delivering, based on the thermal data, a second amount of energy to the one or more perivascular tissues.
Clause 26. The method of any of clauses 20 to 25, further comprising classifying at least one of the one or more perivascular tissues based on the spatial or temporal temperature distribution of the one or more perivascular tissues.
Clause 27. The method of any of clauses 20 to 26, wherein modulating the temperature of the one or more perivascular tissues comprises delivering energy to or removing energy from the one or more perivascular tissues.
Clause 28. The method of any of clauses 20 to 27, wherein modulating the temperature of the one or more perivascular tissues comprises controlling an energy source to deliver energy to the one or more perivascular tissues to heat the one or more perivascular tissues.
Clause 29. The method of clause 28,
   wherein the energy source comprises a plurality of focal energy sources arranged around the perimeter of the support structure, and
   wherein the energy is delivered to the one or more perivascular tissues using the plurality of focal energy sources.
Clause 30. The method of any of clauses 20 to 29,
   wherein each focal energy source comprises an electrode,
   wherein modulating the temperature of the one or more perivascular tissues comprises delivering, by at least one electrode, an energy field to the one or more perivascular tissues, and
   wherein the method comprises receiving, for each electrode, a distribution of temperatures of perivascular tissue near the electrode representing a thermal field of the electrode.
Clause 31. The method of clause 30, further comprising, in response to determining that the thermal field is dispersed from the respective electrode, adjusting the energy field toward the respective electrode.
Clause 32. The method of clause 30, further comprising, in response to determining that a temperature of the thermal field near the respective electrode is relatively high compared to at least one other electrode, adjusting the energy field toward the respective electrode.
Clause 33. The method of clause 30, further comprising, in response to determining that the thermal field is localized to the electrode, adjusting the energy field away from the respective electrode.
Clause 34. The method of clause 30, further comprising, in response to determining that a temperature of the thermal field near the respective electrode is relatively low compared to at least one other electrode, adjusting the energy field away from the respective electrode.
Clause 35. The method of any of clauses 20 to 34, wherein modulating the temperature of the one or more perivascular tissues comprises modulating a flow rate of blood to the one or more perivascular tissues.
Clause 36. The method of any of clauses 20 to 34,
   wherein the intravascular medical device is a first intravascular medical device positioned in a first vessel, and
   wherein modulating the temperature of the one or more perivascular tissues comprises delivering, from a second intravascular medical device positioned in a second vessel, energy to the one or more perivascular tissues.
Clause 37. The method of clause 36, wherein a distance between the first vessel and the second vessel is less than about three centimeters.
Clause 38. The method of clause 36 or 37, further comprising:
   delivering, by the first intravascular medical device in the first vessel, energy to the one or more perivascular tissues;
   detecting, using the second intravascular medical device in the second vessel, a spatial or temporal distribution of temperatures at or near a wall of the second vessel; and
   generating thermal data representing the spatial or temporal temperature distribution of the temperatures at or near the wall of the first vessel and the spatial or temporal temperature distribution of the temperatures at or near the wall of the second vessel.
Clause 39. The method of any of clauses 20 to 34,
   wherein the intravascular medical device is a first intravascular medical device, and
   wherein modulating the temperature of the one or more perivascular tissues comprises delivering, from a second intravascular medical device positioned in the vessel, energy to the one or more perivascular tissues.
Clause 40. An intravascular medical device assembly, comprising:
   a first intravascular medical device comprising:
      a first support structure defining a longitudinal axis and configured to be positioned within a first vessel of a patient; and
      an energy source coupled to the first support structure, wherein the energy source is configured to deliver energy to one or more perivascular tissues near the first vessel to heat the one or more perivascular tissues; and
   a second intravascular medical device comprising:
      a second support structure defining a longitudinal axis and configured to be positioned within a second vessel of a patient; and
      a plurality of temperature sensors arranged around the perimeter of the second support structure, wherein each of the plurality of temperature sensors is configured to measure a temperature at or near a wall of the second vessel.
Clause 41. The intravascular medical device assembly of clause 40, wherein each of the plurality of temperature sensors is further configured to contact the wall of the second vessel.
Clause 42. The intravascular medical device assembly of clause 41, wherein the second support structure is configured to radially expand from a delivery configuration to a deployed position to cause the plurality of temperature sensors to contact the wall of the second vessel.
Clause 43. The intravascular medical device assembly of any of clauses 40 to 42, wherein the second intravascular medical device further comprises an actuation assembly configured to extend the plurality of temperature sensors through the wall of the second vessel.
Clause 44. The intravascular medical device assembly of any of clauses 40 to 43,
   wherein each of the plurality of temperature sensors corresponds to a particular axial and circumferential position on the second support structure,
   wherein the second intravascular medical device is configured to output a temperature signal that includes a temperature measurement from each of the plurality of temperature sensors, and
   wherein each temperature measurement represents the temperature of the wall of the second vessel at the respective axial and circumferential position on the second support structure.
Clause 45. The intravascular medical device assembly of any of clauses 40 to 44, wherein a spacing of adjacent temperature sensors of the plurality of temperature sensors is less than about 10 mm.
Clause 46. The intravascular medical device assembly of any of clauses 40 to 44, wherein an angular spacing of adjacent temperature sensors of the plurality of temperature sensors around the perimeter of the second support structure is less than or equal to about 90 degrees.
Clause 47. The intravascular medical device assembly of any of clause 40 to 46,
   wherein the energy source comprises a plurality of focal energy sources arranged around a perimeter of the first support structure, and
   wherein each of the plurality of focal energy sources is configured to deliver energy to one or more perivascular tissues near the first vessel to heat the one or more perivascular tissues.
Clause 48. The intravascular medical device assembly of clause 47, wherein at least a portion of the plurality of focal energy sources is configured to:
   deliver a current to the wall of the first vessel; and
   measure an impedance from the wall of the first vessel, wherein the impedance represents an impedance of the one or more perivascular tissues.
Clause 49. The intravascular medical device assembly of clause 47 or 48, wherein each of the plurality of focal energy sources is independently operable.
Clause 50. The intravascular medical device assembly of any of clauses 47 to 49, wherein the plurality of focal energy sources comprises a plurality of radiofrequency electrodes.
Clause 51. The intravascular medical device assembly of any of clauses 47 to 50, further comprising an energy field generator communicatively coupled to the plurality of focal energy sources and configured to control the plurality of focal energy sources to deliver the energy to the one or more perivascular tissues.
Clause 52. The intravascular medical device assembly of clause 51, wherein the energy field generator is configured to, in an imaging mode, control the plurality of focal energy sources to heat the one or more perivascular tissues below an ablation temperature of the one or more perivascular tissues.
Clause 53. The intravascular medical device assembly of clause 51 or 52, wherein the energy field generator is further configured to, in an ablation mode, control the plurality of focal energy sources to heat the one or more perivascular tissues above an ablation temperature of the one or more perivascular tissues.
Clause 54. The intravascular medical device assembly of any of clauses 51 to 53, wherein the energy field generator is configured to:
   receive a temperature signal that includes a temperature measurement from each of the plurality of temperature sensors; and
   modify, based on the temperature signal, the energy delivered to at least one focal energy source of the plurality of focal energy sources.
Clause 55. The intravascular medical device assembly of any of clauses 40 to 54, wherein first support structure comprises an ablation catheter.

1. An intravascular medical device, comprising:
a support structure defining a longitudinal axis and configured to be positioned within a vessel of a patient;
a plurality of focal energy sources arranged around a perimeter of the support structure, wherein each of the plurality of focal energy sources is configured to deliver energy to one or more perivascular tissues near the vessel to heat the one or more perivascular tissues; and
a plurality of temperature sensors arranged around the perimeter of the support structure, wherein each of the plurality of temperature sensors is configured to measure a temperature at or near a wall of the vessel.

2. The intravascular medical device of claim 1, wherein each of the plurality of temperature sensors is further configured to contact the wall of the vessel, and wherein the support structure is configured to radially expand from a delivery configuration to a deployed position to cause the plurality of temperature sensors to contact the wall of the vessel.

3. The intravascular medical device of claim 1, further comprising an actuation assembly configured to extend the plurality of temperature sensors through the wall of the vessel.

4. The intravascular medical device of claim 1, wherein at least a portion of the plurality of focal energy sources comprise a plurality of electrodes configured to:
deliver a current to the wall of the vessel; and
measure an impedance from the wall of the vessel, wherein the impedance represents an impedance of the one or more perivascular tissues.

5. The intravascular medical device of claim 1,
wherein each of the plurality of temperature sensors corresponds to a particular axial and circumferential position on the support structure,
wherein the intravascular medical device is configured to output a temperature signal that includes a temperature measurement from each of the plurality of temperature sensors, and wherein each temperature measurement represents the temperature of the wall of the vessel at the respective axial and circumferential position on the support structure.

6. The intravascular medical device of claim 1, wherein at least one of a spacing of adjacent temperature sensors of the plurality of temperature sensors is less than about 10 mm or an angular spacing of adjacent temperature sensors of the plurality of temperature sensors around the perimeter of the support structure is less than or equal to about 90 degrees.

7. The intravascular medical device of claim 1, further comprising an energy field generator communicatively coupled to the plurality of focal energy sources and configured to control the plurality of focal energy sources to deliver the energy to the one or more perivascular tissues.

8. The intravascular medical device of claim 7, wherein the energy field generator is further configured to, in an ablation mode, control the plurality of focal energy sources to heat the one or more perivascular tissues above an ablation temperature of the one or more perivascular tissues, and wherein the energy field generator is configured to:
receive a temperature signal that includes a temperature measurement from each of the plurality of temperature sensors; and
modify, based on the temperature signal, the energy delivered to at least one focal energy sources of the plurality of focal energy sources.

9. A tissue mapping system, comprising:
an intravascular medical device, comprising:
a support structure defining a longitudinal axis and configured to be positioned within a vessel of a patient; and
a plurality of temperature sensors arranged around a perimeter of the support structure, wherein the wherein each of the plurality of temperature sensors corresponds to a particular axial and circumferential position on the support structure, and wherein each of the plurality of temperature sensors is configured to measure a temperature at or near the wall of the vessel; and
a tissue mapping device comprising processing circuitry configured to receive a temperature signal that includes a temperature measurement from each of the plurality of temperature sensors, wherein each temperature measurement represents the temperature of the wall of the vessel at the respective axial and circumferential position on the support structure.

10. The tissue mapping system of claim 9, wherein the processing circuitry is further configured to generate, based on the one or more temperature measurements, thermal data representing a spatial or temporal temperature distribution of one or more perivascular tissues near the vessel.

11. The tissue mapping system of claim 10, wherein the thermal data includes a spatial or temporal representation of the temperature of the wall of the vessel at the respective axial and circumferential position on the support structure, and wherein the processing circuitry is configured to classify at least one of the one or more perivascular tissues based on the spatial or temporal temperature distribution of the one or more perivascular tissues.

12. A method, comprising:
modulating a temperature of one or more perivascular tissues near a vessel of a patient; and
detecting, using an intravascular medical device positioned in the vessel, a spatial or temporal distribution of temperatures at or near a wall of the vessel,
wherein the intravascular medical device comprises a support structure defining a longitudinal axis and a plurality of temperature sensors arranged around a perimeter of the support structure.

13. The method of claim 12, wherein detecting the spatial or temporal distribution of temperatures comprises:
measuring, by each of the plurality of temperature sensors, a temperature at or near the wall of the vessel; and
generating, based on one or more temperature measurements of the temperature at or near the wall of the vessel, thermal data representing the spatial or temporal temperature distribution of the temperatures.

14. The method of claim 12, further comprising classifying at least one of the one or more perivascular tissues based on the spatial or temporal temperature distribution of the one or more perivascular tissues.

15. The method of claim 12, wherein modulating the temperature of the one or more perivascular tissues comprises delivering energy to or removing energy from the one or more perivascular tissues.

16. An intravascular medical device assembly, comprising:
a first intravascular medical device comprising:
a first support structure defining a longitudinal axis and configured to be positioned within a first vessel of a patient; and
an energy source coupled to the first support structure, wherein the energy source is configured to deliver energy to one or more perivascular tissues near the first vessel to heat the one or more perivascular tissues; and
a second intravascular medical device comprising:
a second support structure defining a longitudinal axis and configured to be positioned within a second vessel of a patient; and
a plurality of temperature sensors arranged around the perimeter of the second support structure, wherein each of the plurality of temperature sensors is configured to measure a temperature at or near a wall of the second vessel.

17. The intravascular medical device assembly of claim 16, wherein each of the plurality of temperature sensors is further configured to contact the wall of the second vessel, and wherein the second support structure is configured to radially expand from a delivery configuration to a deployed position to cause the plurality of temperature sensors to contact the wall of the second vessel.

18. The intravascular medical device assembly of claim 16, wherein the second intravascular medical device further comprises an actuation assembly configured to extend the plurality of temperature sensors through the wall of the second vessel.

19. The intravascular medical device assembly of claim 16, wherein at least one of a spacing of adjacent temperature sensors of the plurality of temperature sensors is less than about 10 mm or an angular spacing of adjacent temperature sensors of the plurality of temperature sensors around the perimeter of the second support structure is less than or equal to about 90 degrees.

20. The intravascular medical device assembly of claim 16,
wherein each of the plurality of temperature sensors corresponds to a particular axial and circumferential position on the second support structure,
wherein the second intravascular medical device is configured to output a temperature signal that includes a temperature measurement from each of the plurality of temperature sensors, and
wherein each temperature measurement represents the temperature of the wall of the second vessel at the respective axial and circumferential position on the second support structure.

21. The intravascular medical device of claim 1, wherein each of the plurality of focal energy sources is independently operable.

22. The intravascular medical device of claim 1, wherein the plurality of focal energy sources comprises a plurality of radiofrequency electrodes.

23. The intravascular medical device of claim 7, wherein the energy field generator is configured to, in an imaging mode, control the plurality of focal energy sources to heat the one or more perivascular tissues below an ablation temperature of the one or more perivascular tissues.

24. The method of claim 12,
wherein each focal energy source comprises a respective electrode,
wherein modulating the temperature of the one or more perivascular tissues comprises delivering, by at least one electrode, an energy field to the one or more perivascular tissues,
the method further comprising receiving, for each electrode, a distribution of temperatures of perivascular tissue near the respective electrode representing a thermal field of the respective electrode, and at least one of:
in response to determining that the thermal field is dispersed from the respective electrode, adjusting the energy field toward the respective electrode;
in response to determining that a temperature of the thermal field near the respective electrode is relatively high compared to at least one other electrode, adjusting the energy field toward the respective electrode;
in response to determining that the thermal field is localized to the respective electrode, adjusting the energy field away from the respective electrode; or
in response to determining that a temperature of the thermal field near the respective electrode is relatively low compared to at least one other electrode, adjusting the energy field away from the respective electrode.
